(19) United States
(12) Patent Application Publication
(10) Pub. No.: US 2010/0285537 A1
(43) Pub. Date: Nov. 11, 2010

(54) SELECTIVE TAGGING OF SHORT NUCLEIC ACID FRAGMENTS AND SELECTIVE PROTECTION OF TARGET SEQUENCES FROM DEGRADATION

(75) Inventor: Bernhard G. Zimmermann, San Mateo, CA (US)

Correspondence Address: Weaver Austin Villeneuve & Sampson LLP, P.O. BOX 70250, OAKLAND, CA, 94612-0250, US

(73) Assignee: FLUIDIGM CORPORATION, South San Francisco, CA (US)

(21) Appl. No.: 12/752,974

(22) Filed: Apr. 1, 2010

Related U.S. Application Data
(60) Provisional application No. 61/166,156, filed on Apr. 2, 2009.
(60) Provisional application No. 61/237,210, filed on Aug. 26, 2009.

(51) Int. Cl. C12P 19/34; C02F 3/34

(52) U.S. Cl. 435/91.2; 435/262

(57) ABSTRACT

Methods are provided for selective tagging of short nucleic acids comprising a short target nucleotide sequence over longer nucleic acids comprising the same target nucleotide sequence. The methods can involve performing one or two cycles of amplification of a sample comprising long nucleic acids and short nucleic acids, each comprising the same target nucleotide sequence with at least two target-specific primers or primer pairs under suitable annealing conditions, wherein the primer pairs comprise: an inner primer or primer pair that can amplify the target nucleotide sequence on long and short nucleic acids (wherein each inner primer comprises a 5′ nucleotide tag; and an outer primer or primer pair that amplifies the target nucleotide sequence on long nucleic acids, but not on short nucleic acids); whereby the amplification after a second cycle produces at least one tagged target nucleotide sequence that comprises two nucleotide tags, one from each inner primer, with the target nucleotide sequence located between the nucleotide tags.

### CROSS-REFERENCE TO RELATED APPLICATIONS

This application claims benefit of and priority to U.S. Ser. No. 61/237,210, filed on Aug. 26, 2009, and to U.S. Ser. No. 61/166,156, filed on Apr. 2, 2009, both of which are incorporated herein by reference in their entirety for all purposes.

### FIELD OF THE INVENTION

This invention pertains to the filed of analytics. In certain embodiments methods are provided of selectively tagging short target nucleic acids in a mixed population of short and long nucleic acids both bearing the target nucleotide sequence.

### BACKGROUND OF THE INVENTION

Non-invasive methods for prenatal diagnosis have attracted the attention of clinicians and researchers. Modern ultrasonography or measurement of the levels of maternal serum markers are routinely used as the primary screening tests for developmental malformations. Invasive procedures based on the genetic analysis of fetal chromosomes or DNA from chorionic villus samples or amniocytes are performed in pregnancies at risk for fetal abnormalities and the results obtained are the gold standard for prenatal diagnosis. Because these methods typically involve a 0.5-2% risk for fetal loss, they are recommended mainly in cases at high risk for fetal genetic or cytogenetic abnormalities. The development of a reproducible, reliable, noninvasive method based on retrieval maternal biological samples would render testing feasible for the general population. Despite intensive investigation, however, a satisfactory, clinically acceptable method has not yet emerged.

### SUMMARY OF THE INVENTION

In certain embodiments methods are provided for depleting a nucleic acid sample of non-target nucleic acids. The methods typically involve denaturing the sample nucleic acids in a reaction mixture; contacting the denatured sample nucleic acids with at least one target-specific primer pair under suitable annealing conditions; conducting a first cycle of extension of any annealed target-specific primer pairs by nucleotide polymerization; and after the first cycle of extension, conducting a first cycle of nuclease digestion of single-stranded nucleic acid sequences in the reaction mixture. In certain embodiments the method additionally involves after the first cycle of nuclease digestion, denaturing the nucleic acids in the reaction mixture; contacting the denatured nucleic acids with at least one target-specific primer pair under suitable annealing conditions; conducting a second cycle of extension of any annealed target-specific primer pairs by nucleotide polymerization; and conducting a second cycle of nuclease digestion of single-stranded nucleic acid sequences in the reaction mixture. In various embodiments the same target-specific primer pair is used to prime each of the first and second cycles of extension. In certain embodiments the nuclease is includes, but is not limited to a single strand-specific 3′ exonuclease, a single strand-specific endonuclease, and a single strand-specific 5′ exonuclease. In certain embodiments the nuclease comprises E. coli Exonuclease I. In various embodiments the target-specific primers comprise dU, rather than dT, and dUTP, rather than dTTP, is present in the reaction mixture. In certain embodiments the methods additionally involve after second cycle of nuclease digestion, contacting the reaction mixture with E. coli uracil-n-glycosylase. In certain embodiments the method is carried out using two or more target-specific primer pairs, where each primer pair is specific for a different target nucleotide sequence. In certain embodiments the methods additionally involve after the second cycle of nuclease digestion, denaturing the nucleic acids in the reaction mixture; contacting the denatured nucleic acids with at least one tag specific primer pair under suitable annealing conditions; and amplifying the corresponding tagged target nucleotide sequence.

In certain embodiments methods are also provided for selective tagging of short nucleic acids comprising a short target nucleotide sequence (molecule) over longer nucleic acids comprising the same target nucleotide sequence. The methods typically involve denaturing sample nucleic acids in a reaction mixture, where the sample nucleic acids comprise long nucleic acids and short nucleic acids, each comprising the same target nucleotide sequence; contacting the denatured sample nucleic acids with at least two target-specific primers or primer pairs under suitable annealing conditions, where the primer pairs comprise an inner primer or primer pair that can amplify the target nucleotide sequence on long and short nucleic acids, where each inner primer comprises a 5′ nucleotide tag; and an outer primer or primer pair that amplifies the target nucleotide sequence on long nucleic acids, but not on short nucleic acids; conducting a first cycle of extension of any annealed primer pairs by nucleotide polymerization; after the first cycle of extension, denaturing the nucleic acids in the reaction mixture; subjecting the reaction mixture to suitable annealing conditions; and conducting a second cycle of extension to produce at least one tagged target nucleotide sequence that comprises two nucleotide tags, one from each inner primer, with the target nucleotide sequence located between the nucleotide tags. In certain embodiments the methods additionally involve after the first cycle of extension, digesting single-stranded nucleic acid sequences in the reaction mixture. In certain embodiments the methods additionally involve after the second cycle of extension, digesting single-stranded nucleic acid sequences in the reaction mixture. In certain embodiments the digestion is carried out by adding, to the reaction mixture, a nuclease comprising a single strand-specific 3′ exonuclease, single strand-specific endonuclease, and/or a single strand-specific 5′ exonuclease. In certain embodiments the nuclease comprises E. coli Exonuclease I. In certain embodiments the at least two target-specific primer pairs are protected against digestion with the nuclease. In certain embodiments the method(s) additionally involve after the digestion, adding additional quantities of the at least two target-specific primer pairs to the reaction mixture. In certain embodiments after the first cycle of extension, any subsequent denaturation is carried out at a sufficiently low temperature to avoid denaturation of any extension product of the outer primer pair. In certain embodiments denaturation temperature is about 80° C. to about 85° C. In various embodiments the short nucleic acid fragments are less than about 300 nucleotides in length. In certain embodiments the distance from each outer primer to the target nucleotide sequence is about 130 nucleotides or greater. In certain embodiments the distance from each outer primer to the target nucleotide sequence is about 130 or about 150 nucleotides to about 200 nucleotides. In certain embodiments the short nucleic acid fragments comprise fetal DNA, and the long nucleic acid fragments comprise maternal DNA. In certain embodiments the sample comprises maternal plasma or urine. In certain embodiments the short nucleic acid fragments comprise tumor DNA, and the long nucleic acids comprise normal DNA. In certain embodiments the sample comprises plasma from a cancer patient. In certain embodiments the method(s) additionally involve subjecting the reaction mixture to one or more cycles of amplification, where annealing is carried out at a sufficiently high temperature that the inner primers will only anneal to tagged target nucleotide sequences. In certain embodiments the method(s) additionally involve contacting the at least one tagged target nucleotide sequence with a tag-specific primer pair under suitable annealing conditions; and amplifying or otherwise detecting and/or quantifying the tagged target nucleotide sequence. In certain embodiments the method(s) additionally involve quantifying the amount of the at least one tagged target nucleotide sequence produced by amplification. In certain embodiments the quantifying comprises subjecting the tagged target nucleotide sequence(s) to digital amplification. In certain embodiments the amplification comprises a preamplification that produces at least one target amplicon. In certain embodiments the preamplification comprises amplifying a tagged reference nucleic acid to produce a reference amplicon. In certain embodiments the digital amplification comprises distributing the preamplified target and reference amplicons into discrete digital amplification mixtures, where each digital amplification mixture, on average, includes no more than one amplicon per mixture; and subjecting the digital amplification mixtures to amplification. In certain embodiments the digital amplification comprises real-time PCR. In certain embodiments the digital amplification comprises endpoint PCR. In certain embodiments the digital amplification comprises: determining the number of reaction mixtures containing amplification product derived from a particular target amplicon; determining the number of reaction mixtures containing amplification product derived from the reference amplicon; and determining the copy number for each target amplicon relative to the reference amplicon. In certain embodiments the target amplicon is derived from fetal DNA. In certain embodiments the target amplicon is derived from tumor DNA. In various embodiments the method is carried out using at least one additional set of inner and outer target-specific primer pairs, when the set is specific for at least one additional target nucleotide sequence. In certain embodiments the additional inner primer pair comprises 5′ nucleotide tags that are different from the initial 5′ nucleotide tags. In certain embodiments the additional inner primer pair comprises 5′ nucleotide tags that are the same as the initial 5′ nucleotide tags. In various embodiments at least two different target nucleotide sequences that are tagged with the same tags are located on the same chromosome. In certain embodiments the amplification is carried out in one or more compartment(s) of a microfluidic device. In various embodiments the microfluidic device is fabricated, at least in part, from an elastomeric material. In certain embodiments the method(s) additionally involve detecting and/or quantifying the tagged short target nucleic acid. In certain embodiments the presence of a target amplicon is determined by ligase detection reaction (LDR), and/or by quantitative real-time polymerase chain reaction (qPCR). In certain embodiments a universal qPCR probe is employed to detect target amplicon(s). In certain embodiments the universal qPCR probe comprises a double-stranded DNA-binding dye. In certain embodiments one or more target-specific qPCR probes is employed to detect target amplicon(s). In certain embodiments the presence of a target amplicon is detected using a fluorogenic nuclease assay. In certain embodiments the presence of a target amplicon is detected using a dual-labeled fluorogenic hydrolysis oligonucleotide probe. In various embodiments the method is performed to determine genotypes at loci corresponding to the target nucleotide sequence. In various embodiments the method is performed to determine copy number at loci corresponding to the target nucleotide sequence. In various embodiments the method is performed to determine the presence or absence of fetal aneuploidy. In various embodiments the method is performed to prepare target nucleotide sequence(s) for sequencing.

In certain embodiments the sample in the methods comprises a genomic DNA sample. In various embodiments then, one or more amplification cycles can be conducted in the presence of an amount of a blocking agent (e.g., a nucleic acid blocking agent that hybridizes to repetitive sequences in the genomic DNA sample) that is sufficient to increase specific amplification of the target nucleic acid. In certain embodiments the blocking agent is selected from the group consisting of tRNA, degenerate oligonucleotide primers, repetitive DNA, bovine serum albumin (BSA), and glycogen. In certain embodiments the blocking agent is present at a concentration in the range of about 0.1 μg/μL to about 40 μg/μL. In certain embodiments the blocking agent comprises tRNA at a concentration in the range of about 1 μg/μL to about 5 μg/μL.

In certain embodiments other aspects of the invention include (1) a method of increasing the specific amplification of a target nucleic acid from a genomic DNA sample and (2) a method of increasing the specific amplification of a plurality of target nucleic acids in a multiplex amplification reaction. In particular embodiments, these methods both entail conducting the amplification in the presence of an amount of a blocking agent sufficient to increase specific amplification of the target nucleic acid. In specific embodiments, the amplification is carried out by polymerase chain reaction (PCR).

Illustrative blocking agents include tRNA, degenerate oligonucleotide primers, repetitive DNA, bovine serum albumin (BSA), and glycogen. In one particular embodiment, the blocking agent is present in the amplification reaction mixture at a concentration in the range of about 0.1 μg/μL to about 40 μg/μL. In certain illustrative embodiments, tRNA is employed as blocking agent at a concentration in the range of about 1 μg/μL to about 5 μg/μL.

### BRIEF DESCRIPTION OF THE DRAWINGS

FIG. 1A and FIG. 1B illustrate that cell free fetal DNA is significantly fragmented as compared to maternal cell free DNA. FIG. 1A: Length distribution of cell free fetal and cell free total DNA (8 samples). FIG. 1B shows the percentage of fetal DNA as a function of amplicon length.

FIG. 2 provides a flow diagram schematically illustrating the protection of target sequences and the deletion of non-target sequences using a nuclease treatment.

FIGS. 3A and 3B schematically illustrate the results of performing one and two cycles of amplification where the template comprises short nucleic acids (e.g., fetal DNA) containing the target sequence (FIG. 3A), or long nucleic acids (e.g., maternal DNA) also containing the target sequence (FIG. 3B).

FIG. 4 provides a flow diagram schematically illustrating various methods of selectively tagging target sequences on short nucleic acids.

FIG. 5 is a simplified diagram of a nanofluidic biochip according suitable for digital PCR.

FIGS. 6A-6C are simplified diagrams of portion of the nanofluidic biochip illustrated in FIG. 5.

FIG. 7 shows the results (counts in one digital PCR panel of 765 reactions per panel) of amplification of fragmented plasmid, linear plasmid genomic DNA, and NTC with inner primers, outer plus inner primers (with the inner at 100 nM and 300 nM).

FIG. 8 shows a heat map illustrating suppression of long nucleic acids and preferential tagging/amplification of short.

FIG. 9 show a plot illustrating suppression of long nucleic acids in favor of short nucleic acids.

FIGS. 10A and 10B shows a heat map providing a comparison of the amplification performed with inner primers only at 100 nM and 300 nM and with inner primers at 100 nM and 300 nM in combination with outer primers at 900 nM. This figure illustrates suppression of long nucleic acids and preferential tagging/amplification of short.

FIG. 11 shows a heat map showing the tagging results for a T21 plasmid diluted in a gDNA background at 1000, 333, 111, 37, 12, and 0 copies. Results are shown for inner primers at 100 nM and 300 nM in combination with outer primers at 900 nM.

FIG. 12 shows heat map showing the tagging results for a T21 plasmid diluted in water and plasma background at 1000, 333, 111, 37, 12, and 0 copies. Results are shown for inner primers at 100 nM and outer primers at 900 nM.

FIG. 13 shows the results of digital PCR on a 12.765 Digital Array commercially available from Fluidigm Corp. (South San Francisco, Calif.). Human genomic DNA was preamplified in the presence of varying amounts of tRNA and then analyzed by digital PCR, as described in Example 5. Specifically, preamplification was performed on human genomic DNA, using protocols described in Qin et al. (2008) Nucleic Acids Res., 36(18): e116 on the GeneAmp PCR system 9700 (Applied Biosystems, CA) in a 25 μL reaction containing 1× PreAmp master mix (Applied Biosystems, CA), 900 nM primers, ˜10 ng of DNA sample and differing amount of tRNA. Samples were diluted and analyzed on the digital array as described in Qin et al., supra. Equal amounts of genomic DNA were used in all panels shown. The upper two panels show the negative controls—preamplification conducted in the absence of tRNA, while the next two pairs of panels show the effects of adding either 2 μg/μL or 3 μg/μL tRNA to the preamplification reaction mix. It is clear that the addition of tRNA increases the intensity of the specific amplification signal and suppresses background.

FIG. 14 shows the effect of adding tRNA to preamplification reaction mixtures on the quality of specific amplification curve. The plots shown in FIG. 14 are from the experiment described in Example 5 and reflect real time PCR plots from the same chip panels shown in FIG. 13. The first panel shows the amplification plot in the absence of tRNA in the preamplification mix, and the second and third panels show the effect when either 2 μg/μL or 3 μg/μL of tRNA was included in the preamplification reaction mix, respectively. The amplification plots confirm the observation from FIG. 13 that the addition of tRNA increases the total amount of specific amplifiable signal, (increase number of hits) and also show that the addition of tRNA improves the quality of amplification (possibly by improving the efficiency of PCR).

### DETAILED DESCRIPTION

The detection of fetal nucleic acid in maternal biological samples (e.g., plasma, urine, etc.) opens possibilities for the noninvasive detection of fetal conditions including, but not limited to rhesus, D status, sex-linked diseases, fetal genotyping (e.g., SNPs), mutation detection (including sequencing), methylation analysis, fetal anomalies such as aneuploidies, etc. Cell-free fetal DNA in maternal plasma or urine can be also used as a diagnostic tool for diseases of pregnancy such as preeclampsia or preterm labor, and the like.

Because of its low concentration and similarity to maternal DNA, the efficient detection and/or isolation of fetal DNA from maternal biological samples, however, has heretofore proven challenging, particularly in the analysis of aneuploidy.

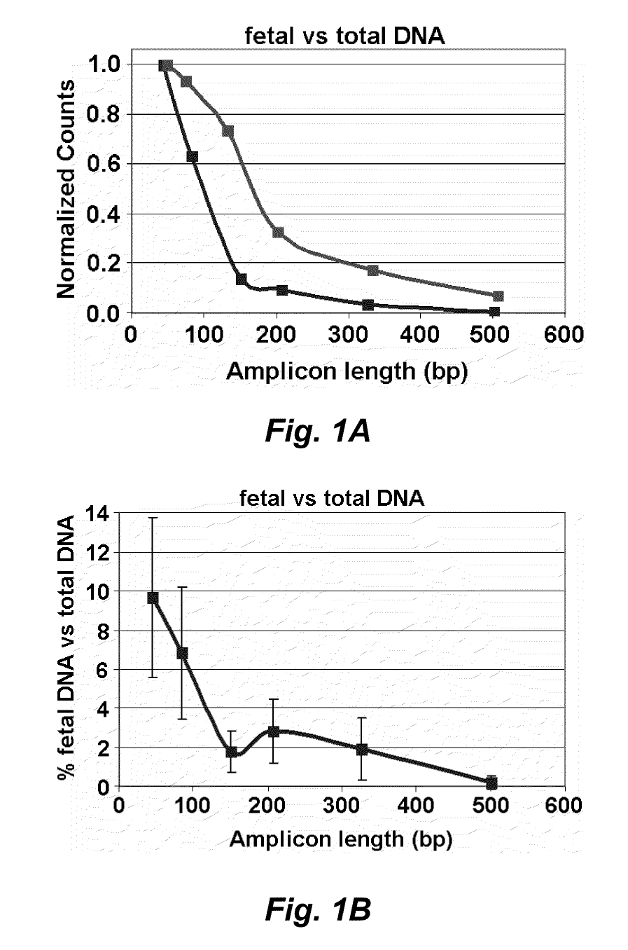

Cell free fetal DNA in maternal biological samples (e.g., samples from pregnant women) has been shown to be fragmented into molecules having a typical length les than about 300 nucleotides, while maternal DNA is predominantly long (see, e.g., FIGS. 1A and 1B). Accordingly methods of preferentially tagging, amplifying and/or isolating short nucleic acid in a population of mixed short and long nucleic acids significantly facilitates the detection, and/or characterization, and/or isolation of fetal DNA, and/or, more generally the enrichment of enrichment of target and/or depletion of non-target DNA.

Accordingly in various embodiments methods are provided that facilitate the detection and/or isolation/amplification of short target nucleic acids (e.g., targets sequences on fetal DNA) while suppressing the tagging and/or amplification of long nucleic acids (e.g., maternal DNA) including those containing the same target nucleotide sequence(s). In certain embodiments the methods involve the amplification of sample nucleic acids using an inner tagged primer or primer pair that amplifies/tags the target sequence(s) and a outer primer or primer pair capable of binding and amplifying longer (e.g., maternal) sequences containing, e.g., the same target nucleotide sequences. As will be explained herein, amplification tagging of the longer sequences is inhibited/blocked.

Also in certain embodiments, methods are provided for protecting target sequences from exonuclease digestion thereby facilitating the elimination in a sample of undesired amplification primers and/or a portion of certain background sequences (e.g., maternal DNA).

While the methods are discussed with respect to the preferential tagging of fetal DNA it will be recognized that they can be applied equally well to the detection and/or isolation of essentially any short target fragment(s) in a mixed population of short and long nucleic acids and is especially useful where both the short and long fragments consist of or contain the target nucleotide sequence.

It is understood that the invention is not limited to the particular methodology, protocols, and reagents, etc., described herein, as these can be varied by the skilled artisan. It is also understood that the terminology used herein is used for the purpose of describing particular illustrative embodiments only, and is not intended to limit the scope of the invention. It also noted that as used herein and in the appended claims, the singular forms “a,” “an,” and “the” include the plural reference unless the context clearly dictates otherwise. Thus, for example, a reference to “a cell” is a reference to one or more cells and equivalents thereof known to those skilled in the art.

The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention.

### DEFINITIONS

The term “adjacent,” when used herein to refer two nucleotide sequences in a nucleic acid, can refer to nucleotide sequences separated by 0 to about 20 nucleotides, more specifically, in a range of about 1 to about 10 nucleotides, or sequences that directly abut one another.

The term “nucleic acid” refers to a nucleotide polymer, and unless otherwise limited, includes known analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; and RNA.

The term nucleic acid encompasses double- or triple-stranded nucleic acid, as well as single-stranded molecules. In double- or triple-stranded nucleic acids, the nucleic acid strands need not be coextensive (i.e., a double-stranded nucleic acid need not be double-stranded along the entire length of both strands).

The term nucleic acid also encompasses any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like.

More particularly, in certain embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing normucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

The term “target nucleic acids” is used herein to refer to particular nucleic acids to be detected in the methods of the invention.

As used herein the term “target nucleotide sequence” refers to a molecule that includes the nucleotide sequence of a target nucleic acid, such as, for example, the amplification product obtained by amplifying a target nucleic acid or the cDNA produced upon reverse transcription of an RNA target nucleic acid.

As used herein, the term “complementary” refers to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Specific hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In particular embodiments, hybridizations are carried out under stringent hybridization conditions. The phrase “stringent hybridization conditions” generally refers to a temperature in a range from about 0° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, 2nd Ed., Vols. 1-3, Cold Spring Harbor Laboratory), both incorporated herein by reference). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in Nucleic Acid Hybridization (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Exemplary stringent conditions suitable for achieving specific hybridization of most sequences are: a temperature of at least about 60° C. and a salt concentration of about 0.2 molar at pH7.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, more typically range from 10 to 30 nucleotides, or even more typically from 15 to 30 nucleotides, in length. Other primers can be somewhat longer, e.g., 30 to 50 nucleotides long. In this context, “primer length” refers to the portion of an oligonucleotide or nucleic acid that hybridizes to a complementary “target” sequence and primes nucleotide synthesis. Short primer molecules generally require cooler temperatures to form sufficiently stable hybrid complexes with the template. A primer need not reflect the exact sequence of the template but must be sufficiently complementary to hybridize with a template. The term “primer site” or “primer binding site” refers to the segment of the target nucleic acid to which a primer hybridizes.

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in certain embodiments, amplification primers used herein are said to “anneal to a sample-specific nucleotide tag.” This description encompasses primers that anneal wholly to the nucleotide tag, as well as primers that anneal partially to the nucleotide tag and partially to an adjacent nucleotide sequence, e.g., a target nucleotide sequence. Such hybrid primers can increase the specificity and/or efficiency of the amplification reaction.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientation in particular embodiments.

A primer pair is said to be “unique” if it can be employed to specifically amplify a particular target nucleotide sequence in a given amplification mixture.

A “probe” is a nucleic acid capable of binding to a target nucleic acid of complementary sequence through one or more types of chemical bonds, generally through complementary base pairing, usually through hydrogen bond formation, thus forming a duplex structure. The probe binds or hybridizes to a “probe binding site.” The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15-20 nucleotides in length).

The primer or probe can be perfectly complementary to the target nucleic acid sequence or can be less than perfectly complementary. In certain embodiments, the primer has at least 65% identity to the complement of the target nucleic acid sequence over a sequence of at least 7 nucleotides, more typically over a sequence in the range of 10-30 nucleotides, and often over a sequence of at least 14-25 nucleotides, and more often has at least 75% identity, at least 85% identity, at least 90% identity, or at least 95%, 96%, 97%. 98%, or 99% identity. It will be understood that certain bases (e.g., the 3′ base of a primer) are generally desirably perfectly complementary to corresponding bases of the target nucleic acid sequence. Primer and probes typically anneal to the target sequence under stringent hybridization conditions.

The term “nucleotide tag” is used herein to refer to a predetermined nucleotide sequence that is added to a target nucleotide sequence. The nucleotide tag can encode an item of information about the target nucleotide sequence, such the identity of the target nucleotide sequence or the identity of the sample from which the target nucleotide sequence was derived. In certain embodiments, such information may be encoded in one or more nucleotide tags, e.g., a combination of two nucleotide tags, one on either end of a target nucleotide sequence, can encode the identity of the target nucleotide sequence. In certain embodiments other non-nucleotide tags can be used instead of or in addition to nucleotide tags. For example, biotin, or other affinity, tags can be incorporated to specifically remove products. In this case the outer or inner primers can be biotin (affinity) tagged to facilitate removal of one amplification product or the other. Other affinity tags can similarly be used. Such affinity tags or “epitope tags” refer to a molecule or domain of a molecule that is specifically recognized by an antibody or other binding partner. The term can also refer to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g., ligands bound by receptors), ligands bound by other ligands to form heterodimers or homodimers, His₆ bound by Ni-NTA, biotin bound by avidin, streptavidin, or anti-biotin antibodies, and the like. Such tags tags are well known to those of skill in the art. Moreover, antibodies specific to a wide variety of tags are commercially available. These include but are not limited to antibodies against the DYKDDDDK (SEQ ID NO:1) epitope, c-myc antibodies (available from Sigma, St. Louis), the HNK-1 carbohydrate epitope, the HA epitope, the HSV epitope, the His₄, His₅, and His₆ epitopes that are recognized by the H is epitope specific antibodies (see, e.g., Qiagen), and the like. In various embodiments it is also possible to use “universal tags” so all targets get the same tag. For example, in certain embodiments, all forward sequences or all reverse sequences In certain embodiments, every assay can be tagged individually or groups of assays can be similarly tagged (e.g. groups of 3, 5, 10, 15, 20 assays, etc.), sequences from the same chromosome can get the same tag, and so forth.

As used herein, the term “encoding reaction” refers to reaction in which at least one nucleotide tag is added to a target nucleotide sequence. Nucleotide tags can be added, for example, by an “encoding PCR” in which the at least one primer comprises a target-specific portion and a nucleotide tag located on the 5′ end of the target-specific portion, and a second primer that comprises only a target-specific portion or a target-specific portion and a nucleotide tag located on the 5′ end of the target-specific portion. For illustrative examples of PCR protocols applicable to encoding PCR, see pending WO Application US03/37808 as well as U.S. Pat. No. 6,605,451. Nucleotide tags can also be added by an “encoding ligation” reaction that can comprise a ligation reaction in which at least one primer comprises a target-specific portion and nucleotide tag located on the 5′ end of the target-specific portion, and a second primer that comprises a target-specific portion only or a target-specific portion and a nucleotide tag located on the 5′ end of the target specific portion. Illustrative encoding ligation reactions are described, for example, in U.S. Patent Publication No. 2005/0260640, which is hereby incorporated by reference in its entirety, and in particular for ligation reactions.

As used herein an “encoding reaction” produces a “tagged target nucleotide sequence,” which includes a nucleotide tag linked to a target nucleotide sequence.

The term “sample-specific” nucleotide tag is used herein to refer to a nucleotide tag that encodes the identity of the sample of the target nucleotide sequence to which the tag is, or becomes, linked in an encoding reaction.

As used herein with reference to a portion of a primer, the term “target-specific nucleotide sequence” refers to a sequence that can specifically anneal to a target nucleic acid or a target nucleotide sequence under suitable annealing conditions.

A “common” sample-specific nucleotide tag refers to a tag having a specific nucleotide sequence that is, or becomes, linked to all target nucleotide sequences produced during an encoding reaction, such that all tagged target nucleotide sequences produced from a given sample are each identified by a tag having the same sequence.

The phrase “a distinct set of forward and reverse primers” refers to a set of primers that is distinguishable from any other sets of primers employed in an assay. Such a set of primers can be used to introduce sample-specific nucleotide tags.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al. (1996) J. Clin. Micro. 34: 501-507; The Nucleic Acid Protocols Handbook, R. Rapley, ed., Humana Press, Totowa, N.J. (2002); Abramson et al. (1993) Curr Opin Biotechnol. 4(1): 41-47, U.S. Pat. Nos. 6,027,998 BS 6,605,451, PCT Publication Nos: WO 97/31256 and WO 01/92579; Day et al. (1995) Genomics, 29(1): 152-162, Ehrlich et al. (1991) Science 252: 1643-1650; Innis et al.,(1990) PCR Protocols: A Guide to Methods and Applications, Academic Press; Favis et al. (2000) Nature Biotechnol., 18: 561-564; Rabenau et al. (2000) Infection 28: 97-102; Belgrader et al. (1995) Development of a Multiplex Ligation Detection Reaction DNA Typing Assay, Sixth International Symposium on Human Identification, (available on the world wide web at: promega.com/geneticidproc/ussymp6proc/blegrad.html-); LCR Kit Instruction Manual, Cat. #200520, Rev. #050002, Stratagene, 2002; Barany (1991) Proc. Natl. Acad. Sci., USA, 88: 188-193; Bi and Sambrook (1997) Nucl. Acids Res. 25: 2924-2951; Zirvi et al. (1999) Nucl. Acid Res. 27: e40i-viii; Dean et al. (2002) Proc. Natl. Acad. Sci., USA, 99: 5261-5266; Barany and Gelfand (1991) Gene 109: 1-11; Walker et al. (1992) Nucl. Acid Res. 20: 1691-1696; Polstra et al. (2002) BMC Inf Dis. 2: 18; Lage et al. (2003) Genome Res. 13(2): 294-307; Landegren et al. (1988) Science 241: 1077-1080; Demidov (2002) Expert Rev Mol. Diagn. 2(6): 542-548; Cook et al. (2003) J. Microbiol. Meth. 53(2): 165-174; Schweitzer et al., (2001) Curr. Opin. Biotechnol. 12(1): 21-27; U.S. Pat. Nos. 5,830,711; 6,027,889; 5,686,243; PCT Publication Nos: WO 00/56927 A3, and WO 98/03673 A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

The term “qPCR” is used herein to refer to quantitative real-time polymerase chain reaction (PCR), which is also known as “real-time PCR” or “kinetic polymerase chain reaction.”

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, and the like. Reagents for enzyme reactions include, for example, substrates, cofactors, buffer, metal ions, inhibitors, and activators.

The term “tag-specific PCR probe” is used herein to refer to a PCR probe that specifically anneals to a nucleotide tag (template). In certain embodiments the tag specific PCR probe will amplify the tag and in certain embodiments, additionally a nucleotide sequence attached to that tag.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation at a wavelength greater than or equal 340 nm.

The term “fluorescent dye,” as used herein, generally refers to any dye that emits electromagnetic radiation of longer wavelength by a fluorescent mechanism upon irradiation by a source of electromagnetic radiation, such as a lamp, a photodiode, or a laser.

The term “elastomer” has the general meaning used in the art. Thus, for example, Allcock et al. (Contemporary Polymer Chemistry, 2nd Ed.) describes elastomers in general as polymers existing at a temperature between their glass transition temperature and liquefaction temperature. Elastomeric materials exhibit elastic properties because the polymer chains readily undergo torsional motion to permit uncoiling of the backbone chains in response to a force, with the backbone chains recoiling to assume the prior shape in the absence of the force. In general, elastomers deform when force is applied, but then return to their original shape when the force is removed.

A “polymorphic marker” or “polymorphic site” is a locus at which nucleotide sequence divergence occurs. Illustrative markers have at least two alleles, each occurring at frequency of greater than 1%, and more typically greater than 10% or 20% of a selected population. A polymorphic site may be as small as one base pair. Polymorphic markers include restriction fragment length polymorphism (RFLPs), variable number of tandem repeats (VNTR's), hypervariable regions, minisatellites, dinucleotide repeats, trinucleotide repeats, tetranucleotide repeats, simple sequence repeats, deletions, and insertion elements such as Alu. The first identified allelic form is arbitrarily designated as the reference form and other allelic forms are designated as alternative or variant alleles. The allelic form occurring most frequently in a selected population is sometimes referred to as the wild type form. Diploid organisms may be homozygous or heterozygous for allelic forms. A diallelic polymorphism has two forms. A triallelic polymorphism has three forms.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

Enhancing Target Sequence Populations in a Sample of Mixed Length Nucleic acids.

In General

Methods are provided for enhancing a nucleic acid sample for target sequences of interest (e.g. fetal DNA) and/or selectively tagging those sequences.

Accordingly in certain embodiments, methods are provided for protecting target sequences from exonuclease digestion thereby facilitating the elimination in a sample of undesired amplification primers and/or a portion of certain background sequences (e.g., maternal DNA).

Methods are also provided for selectively tagging short (e.g., fetal DNA) sequences in a sample comprising long and short nucleic acids by using inner tagged forward and reverse primers (one or both tagged) in combination with outer primers in a nucleic acid amplification (e.g., PCR) mix. As explained below, shorter (e.g., fetal) target nucleic acids are amplified and tagged while the amplification of longer (e.g., maternal nucleic acid sequences) is suppressed by one or more mechanisms including blocking of extension of the inner primers by prior annealing and extension of the outer primers, TaqMan 5′ endonuclease digestion of the inner primer and/or its extension product by extension of the outer primer, and/or displacement of the inner tagged product and exonuclease digestion after amplification cycle 1 or 2.

Selective Protection of Target Sequences from Enzymatic Degradation.

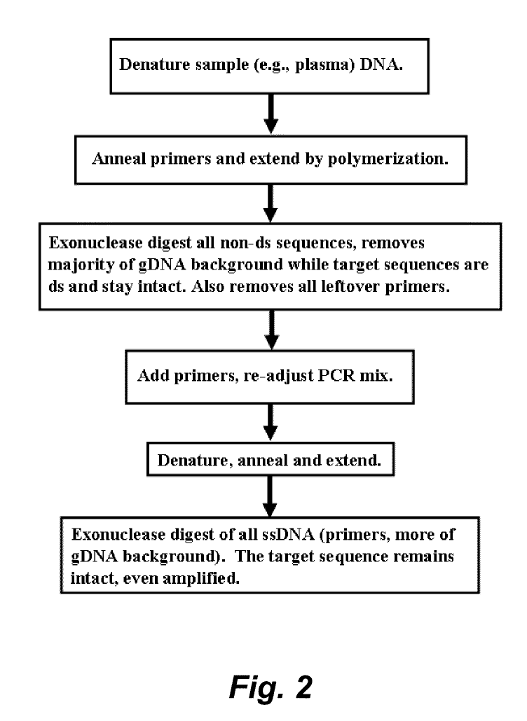

In certain embodiments methods are provided for the selective protection of target nucleic acid sequences from A flow chart schematically illustrating certain embodiments of these methods is provided in FIG. 2. Accordingly, in certain embodiments, the methods comprise denaturing sample nucleic acids in a reaction mixture; contacting the denatured sample nucleic acids with at least one target-specific primer pair under suitable annealing conditions; conducting a first cycle of extension of any annealed target-specific primer pairs by nucleotide polymerization; and after the first cycle of extension, conducting a first cycle of nuclease digestion of single-stranded nucleic acid sequences in the reaction mixture. In various embodiments the methods can further involve denaturing the nucleic acids in the reaction mixture after the first cycle of nuclease digestion; contacting the denatured nucleic acids with at least one target-specific primer pair under suitable annealing conditions; conducting a second cycle of extension of any annealed target-specific primer pairs by nucleotide polymerization; and conducting a second cycle of nuclease digestion of single-stranded nucleic acid sequences in the reaction mixture. The process can optionally be repeated for additional cycles as required. In certain embodiments the same target-specific primer pair is used to prime each of the first and second cycles of extension, while in other embodiments, different target-specific primer pairs are used for the first and second cycle. Any of a variety of nucleases that preferably digest single stranded nucleic acids can be used. Suitable nucleases include for example a single strand-specific 3′ exonuclease, a single strand-specific endonuclease, a single strand-specific 5′ exonuclease, and the like. In certain embodiments the nuclease comprises E. coli Exonuclease I. In certain embodiments the nuclease comprises a reagent such as ExoSAP-IT®. ExoSAP-IT® utilizes two hydrolytic enzymes, Exonuclease I and Shrimp Alkaline Phosphatase, together in a specially formulated buffer to remove unwanted dNTPs and primers from PCR products. Exonuclease I removes residual single-stranded primers and any extraneous single-stranded DNA produced in the PCR. Shrimp Alkaline Phosphatase removes the remaining dNTPs from the PCR mixture. In certain embodiments ExoSAP-IT is added directly to the PCR product and incubated at 37° C. for 15 minutes. After PCR treatment, ExoSAP-IT® is inactivated simply by heating, e.g., to 80° C. for 15 minutes.

In certain embodiments the target-specific primers comprise dU, rather than dT, and dUTP, rather than dTTP, is present in the reaction mixture. In certain embodiments the methods additionally comprise contacting the reaction mixture with E. coli Uracil-N-Glycosylase after the second cycle of nuclease digestion. In one illustrative embodiment, the method is carried out using two or more target-specific primer pairs, where each primer pair is specific for a different target nucleotide sequence. In various embodiments, particular, where the target specific primers introduced nucleotide tags, the method can involve after the second cycle of nuclease digestion, denaturing the nucleic acids in the reaction mixture; contacting the denatured nucleic acids with at least one target (e.g., tag) specific primer pair under suitable annealing conditions; and amplifying the corresponding (e.g., tagged) target nucleotide sequence.

Selective Tagging of Short Target Sequences.

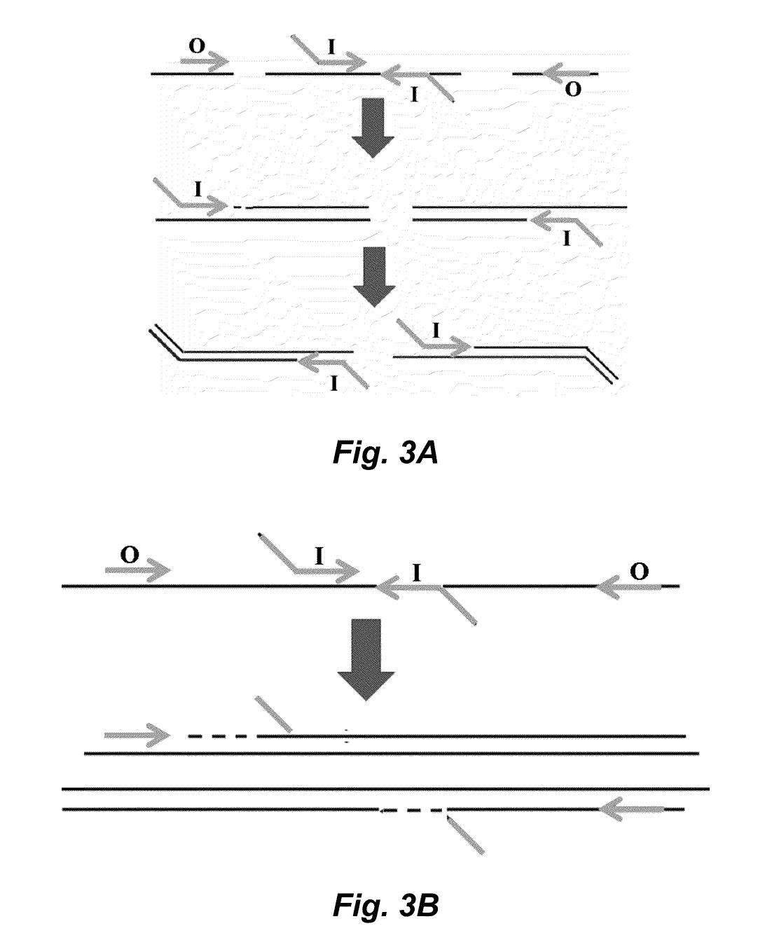

In certain embodiments methods are provided for selectively tagging short target sequences (e.g., cell free fetal DNA) in a mixed population of short and long nucleic acids (e.g., cell free DNA obtained from maternal plasma). In various embodiments the method typically involves performing a nucleic acid amplification using a set of nested primers comprising inner primers and outer primers (see, e.g., FIGS. 3A and 3B). In various embodiments one or both of the inner can be tagged to thereby introduce a tag onto the target amplification product. FIGS. 3A and 3B schematically illustrate the performing one and two cycles of amplification where the template comprises short nucleic acids (e.g., fetal DNA) containing the target sequence (FIG. 3A), or long nucleic acids (e.g., maternal DNA) also containing the target sequence) (FIG. 3B).

As illustrated in FIG. 3A the outer primers (labeled “O” in the figure) do not anneal on the short fragments (e.g., fetal DNA) that carry the (inner) target sequence. The inner primers (labeled “I” in the figure) anneal to the short fragments and generate an amplification product that carries a tag and the target sequence. After 2 cycles a short double stranded fragment generates two double stranded products (which are 3′-exonuclease resistant). One strand of each of these carries both tags (where both primers were tagged).

At the same time, tagging of the long fragments (e.g., maternal DNA) is inhibited as illustrated in FIG. 3B. This occurs through a combination of mechanisms. First, the extension of the inner primers can be blocked by the prior annealing and extension of the outer primer. Second, the extension of the outer primer can lead to cleavage of the tag from the already annealed inner primer. The third possibility is that the inner primers' extension product is displaced but intact. The result is that after two cycles, target sequences on the short nucleic acids (e.g., cell free fetal DNA) are tagged, while the longer nucleic acids (e.g., cell free maternal DNA), even those containing the target nucleotide sequence, are not tagged. Moreover, the tagged amplification products from the short sequences are double stranded and thereby 3′-exonuclease resistant.

At this point, enrichment for tagged target sequences (e.g., fetal DNA) can readily be accomplished by any of a variety of methods. For example, an exonuclease digestion can be performed (e.g., as described above) to digest all non-double stranded sequences including extension products of displaces inner primers. This removes the majority of genomic DNA background, while the target sequence are double stranded and stay intact. This also removes substantially all leftover primers.

In certain embodiments after the first cycle, and preferably after second cycle it is possible to directly continue thermocycling (e.g., without exonuclease digestion), but increasing the annealing temperature (e.g., from 60° C. to 72° C.). As a consequence, the inner primers will amplify only sequences that are tagged. The primers cannot bind to untagged target sequences.

In certain embodiments the denaturation temperature is selected to avoid melting of the long DNA amplification product(s). This can be applied right at the first cycle or after a limited amount of amplification rounds, when the short fragments have formed a PCR product that will melt at low temperatures (e.g., 70° C.-80° C.).

In certain embodiments the primers used for further amplification (e.g., after the first cycle and preferably after the second cycle) are specific to the two tags and not to the target sequences.

The resulting amplified tagged target sequences can be analyzed by any convenient methods. Such methods include, for example several modes of PCR (or other amplification methods). Several choices of how to encode target sequences by tagging can be selected. Straightforward is digital PCR. To multiplex several targets (e.g. per chromosome 21), these targets can be encoded with the same two tags. For each chromosome one could use only one primer pair in the PCR reaction.

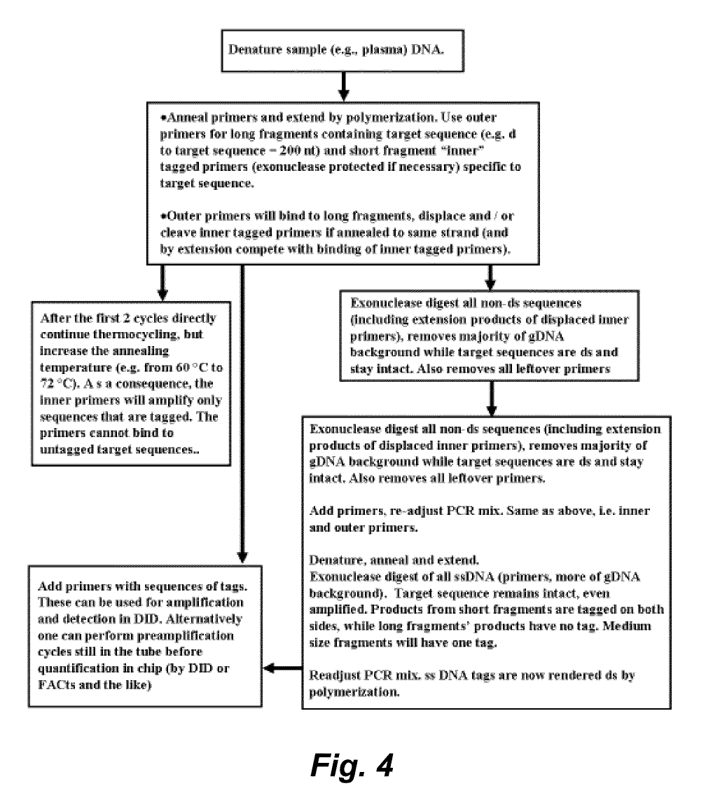

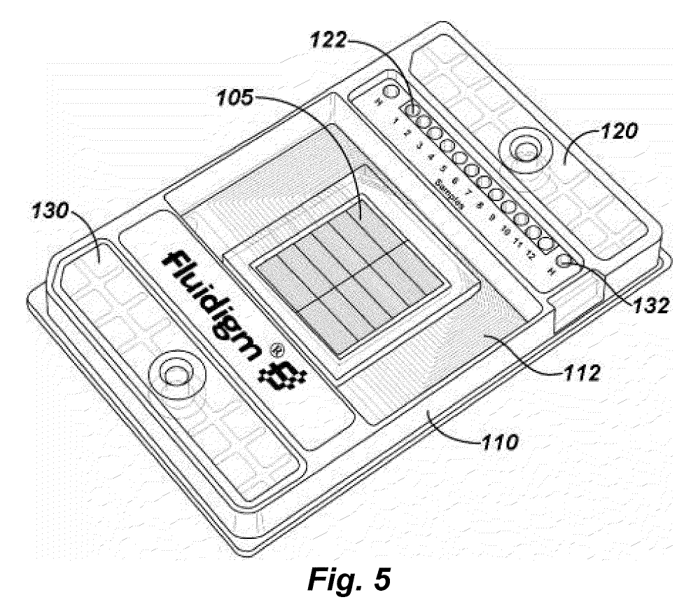

Accordingly, in certain embodiments, methods are provided for selective tagging of short nucleic acids comprising a short target nucleotide sequence (nucleic acid) over longer nucleic acids comprising the same target nucleotide sequence. Various embodiments are schematically illustrated in the flowchart provided in FIG. 4. As illustrated therein, in various embodiments the method involves denaturing sample nucleic acids in a reaction mixture, where the sample nucleic acids comprise long nucleic acids and short nucleic acids, each comprising the same target nucleotide sequence. The denatured sample nucleic acids are contacted with one or preferably at least two target-specific primer pairs under suitable annealing conditions, where the primer pairs comprise an inner primer pair (one or both carrying a nucleotide tag, e.g., a 5′ nucleotide tag) that can amplify the target nucleotide sequence on long and short nucleic acids; and an outer primer pair that amplifies the target nucleotide sequence on long nucleic acids, but not on short nucleic acids. A first cycle of extension is conducted for any annealed primer pairs by nucleotide polymerization. After the first cycle of extension, the nucleic acids in the reaction mixture are denatured, the reaction mixture is subjected to suitable annealing conditions; and a second cycle of extension is conducted to produce at least one tagged target nucleotide sequence that comprises two nucleotide tags, one from each inner primer, with the target nucleotide sequence located between the nucleotide tags. It will be recognized that in certain embodiments, one use primers for only one strand in a simple mode, or for one strand per cycle.)

In certain embodiments, the method can additionally involve digesting single-stranded nucleic acid sequences in the reaction mixture after the first and/or the second cycle. In certain embodiments the digestion can by the use of an endonuclease (e.g., single strand-specific 3′ exonuclease, single strand-specific endonuclease, a single strand-specific 5′ exonuclease, a combination of exonuclease alkaline phosphatase, etc.), e.g., as described above. The nuclease treatment digests substantially all non-double stranded sequences (including remaining primers, extension products of displaced inner primers, etc.), removes a substantial portion of gDNA background while leaving intact the double stranded target sequences.

In certain embodiments, as a substitute for the digestion, or in addition to the digestion, the method additionally comprises adding additional quantities the same or different target-specific primer pairs to the reaction mixture and performing one or more amplification cycles to preferentially amplify the tagged target sequences.

In certain embodiments after the first cycle of extension, any subsequent denaturation is carried out at a sufficiently low temperature (e.g. about 80° C. to about 85° C.) to avoid denaturation of any extension product of the outer primer pair.

In certain preferred embodiments, the method additionally comprises subjecting the reaction mixture to one or more cycles of amplification, wherein annealing is carried out at a sufficiently high temperature that the inner primers will only anneal to tagged target nucleotide sequences. This can be during the first to cycles and/or after the first two amplification cycles.

In certain embodiments the method(s) additionally involve contacting the at least one tagged target nucleotide sequence with a tag-specific primer pair under suitable annealing conditions; and amplifying the tagged target nucleotide sequence or using other modes of detection and/or quantification, e.g. as described herein. In certain embodiments the method further involves detecting and/or quantifying the amount of at least one tagged target nucleotide sequence produced by amplification (e.g., via digital PCR (dPCR)).

In certain embodiments the “short” nucleic acid fragments are less than about 500 nucleotides, preferably less than about 400, more preferably less than about 350 nucleotides, and most preferably about 300 nucleotides or shorter (e.g., 250 nt, 200 nt, etc.).

While the methods described herein can be used with essentially any nucleic acid sample comprising long and short nucleic acids (nucleic acid molecules), in certain embodiments, the short nucleic acids comprise fetal nucleic acids (e.g., cell free fetal DNA from maternal plasma or urine), while the long nucleic acids comprise maternal nucleic acids (e.g., cell free maternal DNA from plasma or urine). In various embodiments the nucleic acid are derived from a maternal biological sample (e.g., a biological sample from a pregnant mammal (e.g., human) comprising maternal plasma, maternal urine, amniotic fluid, etc.). In certain embodiments the nucleic acids are derived from a biological sample from a mammal (e.g., a human or non-human mammal) having, suspected of having, or at risk for, a pathology or congenital disorder characterized by a nucleic acid abnormality (e.g., aneuploidy, fragmentation, amplification, deletion, single-nucleotide polymorphism, translocation, chromosomal rearrangement or resorting, etc.). In certain embodiments the nucleic acids are derived from a biological sample from a mammal (e.g., a human or non-human mammal) having, suspected of having, or at risk for a cancer. In certain embodiments, the short nucleic acid fragments comprise tumor or metastatic cell DNA, and the long nucleic acids comprise normal DNA.

In certain embodiments the method can be used to determine linkage of two sequence that are relatively neighboring. For example, if an upstream SNP has, for example a “G” nucleotide and the suppression primer(s) are designed to bind to this sequence then amplification of this SNP is suppressed. If the base is an A, the primers bind inefficiently and don't suppress indicating the presence of the A form sequence.

In various embodiments the inner and outer primers are designed/selected so the distance from outer primers to the target nucleotide sequence (measured as the number of nucleotides between the 5′ ends and thereby including the length of both primers) ranges from about 50, 80, 100, 120, 130, 140, or 150 nucleotides or greater. In certain embodiments, the distance from outer primers to the target nucleotide ranges from about 50, 80, 100, 120, 130, 140, or 150 nucleotides to about 400, 350, 300, 250, or 200 nuclides. For selectively tagging fetal versus maternal cell free nucleic acids, the distance from each outer primer to the target nucleotide sequence is greater than about 130 nucleotides, and typically ranges from about 150 to about 200 nucleotides.

It will be recognized that, in certain embodiments, a large number of different target sequences (e.g., 2 or more, 3 or more, 5 or more, 10 or more, 15 or more, 20 or more, 50 or more, 100 or more per chromosome or other template(s)), can be tagged. Moreover using various tagging strategies, different amplification produces are readily discriminated thereby permitting the methods to be highly multiplexed.

In certain embodiments, fetal aneuploidy via Cts can be determined using for example tag-specific primers for pre-amplification (e.g. one primer pair for preamp after 2 tagging cycles), and then again using target specific primers for real-time PCR, e.g., in a chip.

In certain embodiments it is contemplated to apply digital PCR (dPCR) or amplification and dPCR or fetal aneuploidy via CTS to the tagged short fragments. In certain illustrative embodiment the methods are not only useful for determining/detecting fetal aneuploidy but also for fetal genotyping (SNPs), mutation detection (including sequencing), methylation analysis, and the like

It will be appreciated that the methods and applications described herein are illustrative and not limiting. Using the teachings provided herein, other variants and other applications will be available to one or ordinary skill in the art.

### Sample Nucleic Acids

Preparations of nucleic acids (“samples”) can be obtained from biological sources and prepared using conventional methods known in the art. In particular, DNA or RNA useful in the methods described herein can be extracted and/or amplified from any source, including bacteria, protozoa, fungi, viruses, organelles, as well higher organisms such as plants or animals, particularly mammals, and more particularly humans. Suitable nucleic acids can also be obtained from environmental sources (e.g., pond water), from man-made products (e.g., food), from forensic samples, and the like. Nucleic acids can be extracted or amplified from cells, bodily fluids (e.g., blood, a blood fraction, urine, saliva, cerebrospinal fluid, etc.), or tissue samples by any of a variety of standard techniques. Exemplary samples include samples of plasma, serum, spinal fluid, lymph fluid, peritoneal fluid, pleural fluid, oral fluid, and external sections of the skin; samples from the respiratory, intestinal genital, and urinary tracts; samples of tears, saliva, blood cells, stem cells, or tumors. For example, samples of fetal DNA can be obtained from an embryo, from cord blood, from maternal blood (e.g., plasma), from maternal urine, and the like. Samples can be obtained from live or dead organisms or from in vitro cultures. In certain embodiments illustrate samples can include single cells, paraffin-embedded tissue samples, and biopsies (e.g., needle or surgical biopsies). In certain embodiments nucleic acid samples useful in the invention can also be derived from one or more nucleic acid libraries, including cDNA, cosmid, YAC, BAC, P1, PAC libraries, and the like.

Nucleic acids of interest can be isolated using methods well known in the art, with the choice of a specific method depending on the source, the nature of nucleic acid, and similar factors. The sample nucleic acids need not be in pure form, but are typically sufficiently pure to allow the amplification steps of the methods of the invention to be performed. Where the target nucleic acids are RNA, the RNA can be reversed transcribed into cDNA by standard methods known in the art and as described in Sambrook, J., Fritsch, E. F., and Maniatis, T., Molecular Cloning: A Laboratory Manual. Cold Spring Harbor Laboratory Press, NY, Vol. 1, 2, 3 (1989), for example. The cDNA can then be analyzed according to the methods of the invention.

### Target Nucleic Acids

Essentially any target nucleic acid can be selectively protected from enzymatic degradation according to the methods described herein. In various embodiments preferred target nucleic acids are typically long enough to form a stable hybrid duplex in the presence of a reaction mixture containing the various nucleases used in the method. In certain embodiments the target nucleic acid is at least 5, 8, 10, 15, 20, 25, 30, 35, 40, 45, 50, 6,0, 70, 80, 90, 100, 150, 200, 250, or 300 nucleotides in length. In certain embodiments, protection can be achieved by binding of the primer even without extension. For example, where the primer binds to the 3′ end the nuclease digests from 3′ end up to the primer. Downstream there is no digestion. In a 3′ to 5′ digestion oen can two primers to flank and protect the sequence.

Similarly, essentially any target nucleic acid in a mixed population of short and long nucleic acids can be tagged, and/or amplified, and/or detected/quantified using the methods described herein. The methods are particularly well suited to mixed populations of long and short nucleic acids where both the long and short nucleic acids contain the target nucleotide sequence.

The short target nucleic acids can be of any length sufficient long to permit amplification. In various embodiments the long nucleic acids are typically of sufficient length to permit the annealing and extension of primers where the primers anneal to a segment of the long nucleic acid that is not amplified by the inner primers that amplify the short target sequence. In various embodiments the longer nucleic acid is preferably at least 8, 10, 12, 15, 20, 25, 30, 35, 40, 45, 50, 55, 60, 70, 80, 100, 120, 130, 150, 180, or 200 nucleotides longer than the on one or both sides (3′ and/or 5′) of the short target sequences. In certain embodiments the long nucleic acids are at least 1.1, 1.2. 1.5, 2.0, 3.0, 4.0, 5, 0, 8.0, or 10 fold longer than the short nucleic acids. In certain embodiments the “short” nucleic acid fragments are less than about 500 nucleotides, preferably less than about 400, more preferably less than about 350 nucleotides, and most preferably about 300 nucleotides or shorter (e.g., 250 nt, 200 nt, etc.).

In typical embodiments, at least some nucleotide sequence information will be known for the target nucleic acids as we as the “long” nucleic acids. More typically, sufficient sequence information is generally available for each end of a given target nucleic acid and the long nucleic acids to permit design of suitable inner and outer amplification primers.

The targets can include, for example, nucleic acids associated with pathogens, such as viruses, bacteria, protozoa, or fungi; RNAs, e.g., those for which over- or under-expression is indicative of disease, those that are expressed in a tissue- or developmental-specific manner; or those that are induced by particular stimuli; DNA reverse transcribed from RNAs; genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations). Also of particular interest are fetal nucleic acids including, but not limited to nucleic acids characterized by aneuploidies or other chromosomal abnormalities (e.g., translocations, amplifications, deletions, and the like) as well as particular polymorphisms (e.g., SNPs, alleles, haplotypes, etc.).

### Primer Design

Primers suitable for nucleic acid amplification are sufficiently long to prime the synthesis of extension products in the presence of the agent for polymerization. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 10 or 15 to about 30 nucleotides, although it may contain more or fewer nucleotides. The primers desirably have sufficiently complementary to selectively anneal to their respective strands and form stable duplexes. One skilled in the art knows how to select appropriate primer pairs to amplify the target nucleic acid of interest.

For example, PCR primers can be designed by using any commercially available software or open source software, such as Primer3 (see, e.g., Rozen and Skaletsky (2000) Meth. Mol. Biol., 132: 365-386; www.broad.mit.edu/node/1060, and the like) or by accessing the Roche UPL website. The amplicon sequences are input into the Primer3 program with the UPL probe sequences in brackets to ensure that the Primer3 program will design primers on either side of the bracketed probe sequence.

Primers can be prepared by any suitable method, including, for example, cloning and restriction of appropriate sequences or direct chemical synthesis by methods such as the phosphotriester method of Narang et al. (1979) Meth. Enzymol. 68: 900-99; the phosphodiester method of Brown et al. (1979) Meth. Enzymol. 68: 109-151; the diethylphosphoramidite method of Beaucage et al. (1981) Tetra. Lett., 22: 1859-1862; the solid support method of U.S. Pat. No. 4,458,066 and the like, or can be provided from a commercial source.

Primers can be purified by using a Sephadex column (Amersham Biosciences, Inc., Piscataway, N.J.) or other methods known to those skilled in the art. Primer purification may improve the sensitivity of the methods of the invention.

It is also noted that forward and reverse primers need not necessarily be the same length. Similarly, the “inner” and “outer” primers described herein can be different lengths and thereby facilitate different melting temperatures for the inner and outer hybridizations. Thus, for example, in certain embodiments, the outer primers are longer than the inner primers. In one illustrative embodiment, a PCR reaction can be performed at 65° for one minute where the outer primers bind, but the inner primers don't, down to 60° where the inner primers anneal.

Detection and/or Quantification and/or Characterization of Target Sequences.

In certain embodiments the methods of selective tagging of short target sequences and/or the methods of electively protecting target sequences from enzymatic degradation are capable of providing significant quantities of the desired target sequence(s) for subsequent analysis. Accordingly, in various embodiments, essentially any convenient method can be used to detect and/or quantify and/or characterize the target sequence(s).

Digital Amplification and Optional Preamplification.

In particular embodiments, tagged “short” target nucleotide sequence(s) can be quantified using a digital amplification method. For discussions of “digital PCR” see, for example, Vogelstein and Kinzler (1999) Proc. Natl. Acad. Sci., USA, 96: 9236-9241, and McBride et al., U.S. Patent Application Publication No. 20050252773, especially Example 5 (each of these publications are hereby incorporated by reference in their entirety, and in particular for their disclosures of digital amplification). Digital amplification methods can make use of certain-high-throughput devices suitable for digital PCR, such as microfluidic devices typically including a large number and/or high density of small-volume reaction sites (e.g., nano-volume reaction sites or reaction chambers). In certain embodiments high-throughput sequencing and/or array technologies are utilized.

In illustrative embodiments, digital amplification is performed using a matrix-type microfluidic device, such as the Digital Array microfluidic devices described below. Digital amplification can entail distributing or partitioning a sample among hundreds to thousands of reaction mixtures disposed in a reaction/assay platform or microfluidic device. In such embodiments, a limiting dilution of the sample is made across a large number of separate amplification reactions such that most of the reactions have no template molecules and give a negative amplification result. In counting the number of positive amplification results, e.g., at the reaction endpoint, one is counting the individual template molecules present in the original sample one-by-one. A major advantage of digital amplification is that the quantitation is independent of variations in the amplification efficiency—successful amplifications are counted as one molecule, independent of the actual amount of product.

In particular embodiments, the methods of the invention are employed in determining the copy number of one or more target nucleic acids in a nucleic acid sample. In specific embodiments, methods and systems described herein can be used to detect copy number variation of a target nucleic acid in the genome of a subject by analyzing the genomic DNA present in a sample derived from the subject. For example, digital amplification can be carried out to determine the relative number of copies of a target nucleic acid and a reference nucleic acid in a sample. In certain embodiments, the genomic copy number is known for the reference nucleic acid (i.e., known for the particular nucleic acid sample under analysis). Alternatively, the reference nucleic acid can be one that is normally present in two copies (and unlikely to be amplified or deleted) in a diploid genome, and the copy number in the nucleic acid sample being analyzed is assumed to be two. For example, useful reference nucleic acids in the human genome include sequences of the RNaseP, β-actin, and glyceraldehyde-3-phosphate dehydrogenase (GAPDH) genes; however, it will be appreciated the invention is not limited to a particular reference nucleic acid.

In various embodiments digital amplification is carried out after the methods described herein for selectively tagging short target sequences and/or for protecting target sequences from enzymatic digestion are performed as described herein. In certain embodiments, these methods are performed in lieu of or in addition to a preamplification of sample nucleic acids. Where the methods are used with a preamplification, the preamplification can be performed before or after these methods. Where performed, preamplification prior to digital amplification is performed for a limited number of thermal cycles (e.g., 5 cycles, or 10 cycles). In certain embodiments, the number of thermal cycles during preamplification can range from about 4 to 15 thermal cycles, or about 4-10 thermal cycles. In specific embodiments the number of thermal cycles can be 3, 4, 5, 6, 7, 8, 9, 10, 11, 12, 13, 14, 15, or more than 15.

As those of skill in the art will appreciate, two or more cycles of the short target tagging amplification methods described above is sufficient to produce tagged target nucleotide sequence(s). When performing digital amplification for copy number determination, at least one target nucleotide sequence and at least one reference nucleotide sequence can be tagged. In certain embodiments, this amplification can be continued for a suitable number of cycles for a typical preamplification step, rendering a separate preamplification step unnecessary. Alternatively, different primers, such as, for example, tag-specific primers could be contacted with the tagged target and reference nucleotide sequences and preamplification carried out. For ease of discussion, the term “preamplification” is used below to describe amplification performed prior to digital amplification and the products of this amplification are termed “amplicons.”

In particular embodiments, preamplification reactions preferably provide quantitative amplification of the nucleic acids in the reaction mixture. That is, the relative number (ratio) of the target and reference amplicons should reflect the relative number (ratio) of target and reference nucleic acids in the nucleic acids being amplified. Methods for quantitative amplification are known in the art. See, e.g., Arya et al., 2005, Basic principles of real-time quantitative PCR, Expert Rev Mol. Diagn. 5(2):209-219. In general, primer pairs and preamplification conditions can be selected to ensure that the amplification efficiencies tagged target and tagged reference nucleotide sequences are similar or approximately equal, in order reduce any bias in the copy number determination. The amplification efficiency of any pair of primers can be easily determined using routine techniques (see e.g., Furtado et al. (2004) “Application of real-time quantitative PCR in the analysis of gene expression.” DNA amplification: Current Technologies and Applications. Wymondham, Norfolk, UK: Horizon Bioscience p. 131-145). If the target and reference nucleotide sequences are tagged with the same tags, under suitable conditions, tag-specific primers can amplify both target and reference nucleotide sequences with similar or approximately equal amplification efficiencies. Further, limiting the number of preamplification cycles (typically to less than 15, usually 10 or less than 10, more usually about 5) greatly mitigates any differences in efficiency, such that the typical differences are likely to have an insignificant effect on the results.

Thus, following preamplification and distribution of the preamplified target and reference amplicons into separate digital amplification mixtures, a proportional number of amplicons corresponding to each sequence will be distributed into the mixtures. After digital amplification, the ratio of target and reference amplification products reflects the original ratio. Therefore, one can determine the number of reaction mixtures containing amplification product derived from the target amplicon and determine the number of reaction mixtures containing amplification product derived from the reference amplicon; and the ratio of these numbers provides the copy number of the target nucleic acid (e.g., the tagged target nucleotide sequence) relative to the reference nucleic acid (e.g., the tagged reference nucleotide sequence).

Digital amplification methods are well known to those of skill in the art. Generally, in digital amplification, identical (or substantially similar) amplification reactions are run on a nucleic acid sample, such as genomic DNA. The number of individual reactions for a given nucleic acid sample may vary from about 2 to over 1,000,000. Typically, the number of reactions performed on a sample is about 100 or greater, more typically about 200 or greater, and even more typically about 300 or greater. Larger scale digital amplification can also be performed in which the number of reactions performed on a sample is about 500 or greater, about 700 or greater, about 765 or greater, about 1,000 or greater, about 2,500 or greater, about 5,000 or greater, about 7,500 or greater, or about 10,000 or greater. The number of reactions performed may also be significantly higher, such up to about 25,000, up to about 50,000, up to about 75,000, up to about 100,000, up to about 250,000, up to about 500,000, up to about 750,000, up to about 1,000,000, or even greater than 1,000,000 assays per genomic sample.

In particular embodiments, the quantity of nucleic acid subjected to digital amplification is generally selected such that, when distributed into discrete reaction mixtures, each individual amplification reaction is expected to include one or fewer amplifiable nucleic acids. One of skill in the art can determine the concentration of target amplicon(s) produced as described above and calculate an appropriate amount for use in digital amplification. More conveniently, a set of serial dilutions of the target amplicon(s) can be tested. For example, the device commercially available from Fluidigm Corp. as the 12.765 Digital Array allows 12 different dilutions to be tested simultaneously. Optionally, a suitable dilution can be determined by generating a linear regression plot. For the optimal dilution, the line should be straight and pass through the origin. Subsequently the concentration of the original samples can be calculated from the plot.

The appropriate quantity of target and reference amplicon(s) can be distributed into discrete locations or reaction wells or chambers such that each reaction includes, for example, an average of no more than about one target amplicon and one reference amplicon per volume. The target and reference amplicon(s) can be combined with reagents selected for quantitative or nonquantitative amplification, prior to distribution or after.

Following distribution, the reaction mixtures are subjected to amplification to identify those reaction mixtures that contain a target and/or amplicon. Any amplification method can be employed, but conveniently, PCR is used, e.g., real-time PCR or endpoint PCR. This amplification can employ any primers capable of amplifying the target and/or reference amplicon(s). Digital amplification can be can be carried out wherein the target and reference amplicons are distributed into sets of reaction mixtures for detection of amplification products derived from one type of amplicon, either target or reference amplicons. In such embodiments, two sets of reaction mixtures, a target set and a reference set, could have distinct primer pairs, one for amplifying target amplicons, and one for amplifying reference amplicons could be used. Amplification product could be detected, for example, using a universal probe, such as SYBR Green, or target- and reference-specific probes, which could be included in all digital amplification mixtures.

The concentration of any target or reference amplicon (copies/μL) is correlated with the number of reaction mixtures that are positive (i.e., amplification product-containing) for that particular amplicon. See copending U.S. application Ser. No. 12/170,414, entitled “Method and Apparatus for Determining Copy Number Variation Using Digital PCR,” which is incorporated by reference for all purposes, and, in particular, for analysis of digital PCR results. Also see Dube et al. (2008) “Mathematical Analysis of Copy Number Variation in a DNA Sample Using Digital PCR on a Nanofluidic Device” PLoS ONE 3(8): e2876. doi:10.1371/journal.pone.0002876, which is incorporated by reference for all purposes and, in particular, for analysis of digital PCR results.

Quantitative Real-Time PCR and Other Detection and Quantitation Methods

Any method of detection and/or quantitation of nucleic acids can be used in the invention to detect and/or quantify amplification products. In one embodiment, PCR (polymerase chain reaction) is used to amplify and/or quantitate target nucleic acids. In other embodiments, other amplification systems or detection systems are used, including, e.g., systems described in U.S. Pat. No. 7,118,910 (which is incorporated herein by reference in its entirety for its description of amplification/detection systems) and Invader assays; PE BioSystems). In certain particular embodiments, real-time quantitation methods are used. For example, “quantitative real-time PCR” methods can be used to determine the quantity of a target nucleic acid present in a sample by measuring the amount of amplification product formed during the amplification process itself

Fluorogenic nuclease assays are one specific example of a real-time quantitation method that can be used successfully in the methods described herein. This method of monitoring the formation of amplification product involves the continuous measurement of PCR product accumulation using a dual-labeled fluorogenic oligonucleotide probe—an approach frequently referred to in the literature as the “TaqMan0 method.” See U.S. Pat. No. 5,723,591; Heid et al., 1996, Real-time quantitative PCR Genome Res. 6:986-94, each incorporated herein by reference in their entireties for their descriptions of fluorogenic nuclease assays. It will be appreciated that while “TaqMan® probes” are the most widely used for qPCR, the invention is not limited to use of these probes; any suitable probe can be used.

Other detection/quantitation methods that can be employed in the present invention include FRET and template extension reactions, molecular beacon detection, Scorpion detection, Invader detection, and padlock probe detection.

FRET and template extension reactions utilize a primer labeled with one member of a donor/acceptor pair and a nucleotide labeled with the other member of the donor/acceptor pair. Prior to incorporation of the labeled nucleotide into the primer during a template-dependent extension reaction, the donor and acceptor are spaced far enough apart that energy transfer cannot occur. However, if the labeled nucleotide is incorporated into the primer and the spacing is sufficiently close, then energy transfer occurs and can be detected. These methods are particularly useful in conducting single base pair extension reactions in the detection of single nucleotide polymorphisms and are described in U.S. Pat. No. 5,945,283 and PCT Publication WO 97/22719.

With molecular beacons, a change in conformation of the probe as it hybridizes to a complementary region of the amplified product results in the formation of a detectable signal. The probe itself includes two sections: one section at the 5′ end and the other section at the 3′ end. These sections flank the section of the probe that anneals to the probe binding site and are complementary to one another. One end section is typically attached to a reporter dye and the other end section is usually attached to a quencher dye. In solution, the two end sections can hybridize with each other to form a hairpin loop. In this conformation, the reporter and quencher dye are in sufficiently close proximity that fluorescence from the reporter dye is effectively quenched by the quencher dye. Hybridized probe, in contrast, results in a linearized conformation in which the extent of quenching is decreased. Thus, by monitoring emission changes for the two dyes, it is possible to indirectly monitor the formation of amplification product. Probes of this type and methods of their use are described further, for example, by Piatek et al. (1998) Nat. Biotechnol. 16: 359-363; Tyagi, and Kramer (1996) Nat. Biotechnol., 14: 303-308; and Tyagi, et al., (1998) Nat. Biotechnol. 16:49-53.

The Scorpion detection method is described, for example, by Thelwell et al. (2000) Nucleic Acids Res., 28: 3752-3761 and Solinas et al. (2001) Nucleic Acids Res., 29(20): e96. Scorpion primers are fluorogenic PCR primers with a probe element attached at the 5′-end via a PCR stopper. They are used in real-time amplicon-specific detection of PCR products in homogeneous solution. Two different formats are possible, the “stem-loop” format and the “duplex” format. In both cases the probing mechanism is intramolecular. The basic elements of Scorpions in all formats are: (i) a PCR primer; (ii) a PCR stopper to prevent PCR read-through of the probe element; (iii) a specific probe sequence; and (iv) a fluorescence detection system containing at least one fluorophore and quencher. After PCR extension of the Scorpion primer, the resultant amplicon contains a sequence that is complementary to the probe, which is rendered single-stranded during the denaturation stage of each PCR cycle. On cooling, the probe is free to bind to this complementary sequence, producing an increase in fluorescence, as the quencher is no longer in the vicinity of the fluorophore. The PCR stopper prevents undesirable read-through of the probe by Taq DNA polymerase.

Invader assays (Third Wave Technologies, Madison, Wis.) are used particularly for SNP genotyping and utilize an oligonucleotide, designated the signal probe, that is complementary to the target nucleic acid (DNA or RNA) or polymorphism site. A second oligonucleotide, designated the Invader Oligo, contains the same 5′ nucleotide sequence, but the 3′ nucleotide sequence contains a nucleotide polymorphism. The Invader Oligo interferes with the binding of the signal probe to the target nucleic acid such that the 5′ end of the signal probe forms a “flap” at the nucleotide containing the polymorphism. This complex is recognized by a structure specific endonuclease, called the Cleavase enzyme. Cleavase cleaves the 5′ flap of the nucleotides. The released flap binds with a third probe bearing FRET labels, thereby forming another duplex structure recognized by the Cleavase enzyme. This time, the Cleavase enzyme cleaves a fluorophore away from a quencher and produces a fluorescent signal. For SNP genotyping, the signal probe will be designed to hybridize with either the reference (wild type) allele or the variant (mutant) allele. Unlike PCR, there is a linear amplification of signal with no amplification of the nucleic acid. Further details sufficient to guide one of ordinary skill in the art are provided by, for example, Neri, et al. (2000) Adv. Nucleic Acid and Protein Analysis 3826: 117-125, and U.S. Pat. No. 6,706,471.

Padlock probes (PLPs) are long (e.g., about 100 bases) linear oligonucleotides. The sequences at the 3′ and 5′ ends of the probe are complementary to adjacent sequences in the target nucleic acid. In the central, noncomplementary region of the PLP there is a “tag” sequence that can be used to identify the specific PLP. The tag sequence is flanked by universal priming sites, which allow PCR amplification of the tag. Upon hybridization to the target, the two ends of the PLP oligonucleotide are brought into close proximity and can be joined by enzymatic ligation. The resulting product is a circular probe molecule catenated to the target DNA strand. Any unligated probes (i.e., probes that did not hybridize to a target) are removed by the action of an exonuclease. Hybridization and ligation of a PLP requires that both end segments recognize the target sequence. In this manner, PLPs provide extremely specific target recognition.

The tag regions of circularized PLPs can then be amplified and resulting amplicons detected. For example, TaqMan® real-time PCR can be carried out to detect and quantitate the amplicon. The presence and amount of amplicon can be correlated with the presence and quantity of target sequence in the sample. For descriptions of PLPs see, e.g., Landegren et al., 2003, Padlock and proximity probes for in situ and array-based analyses: tools for the post-genomic era, Comparative and Functional Genomics 4:525-30; Nilsson et al., 2006, Analyzing genes using closing and replicating circles Trends Biotechnol. 24:83-8; Nilsson et al., 1994, Padlock probes: circularizing oligonucleotides for localized DNA detection, Science 265 :2085-8.

In particular embodiments, fluorophores that can be used as detectable labels for probes include, but are not limited to, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, 6-Fam™ are all available from Applied Biosystems, Foster City, Calif.).

Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670.

In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product.

In particular embodiments, combined thermal cycling and fluorescence detecting devices can be used for precise quantification of target nucleic acids. In some embodiments, fluorescent signals can be detected and displayed during and/or after one or more thermal cycles, thus permitting monitoring of amplification products as the reactions occur in “real-time.” In certain embodiments, one can use the amount of amplification product and number of amplification cycles to calculate how much of the target nucleic acid sequence was in the sample prior to amplification.

According to some embodiments, one can simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target nucleic acid.

According to certain embodiments, one can employ an internal standard to quantitate the amplification product indicated by the fluorescent signal (see, e.g., U.S. Pat. No. 5,736,333).

In various embodiments, employing preamplification and/or tagging of the short target sequences as described herein sufficient nucleotide tags can be added to the target nucleotide sequences, so that the relative copy numbers of the tagged target nucleotide sequences is substantially representative of the relative copy numbers of the target nucleic acids in the sample. For example, preamplification can be carried out for 2-20 cycles to introduce the sample-specific or set-specific nucleotide tags. In other embodiments, detection is carried out at the end of exponential amplification, i.e., during the “plateau” phase, or endpoint PCR is carried out. In this instance, preamplification will normalize amplicon copy number across targets and across samples. In various embodiments, preamplification and/or amplification can be carried out for about: 2, 4, 10, 15, 20, 25, 30, 35, or 40 cycles or for a number of cycles falling within any range bounded by any of these values.

### Use of Blocking Agents During Amplification

In certain embodiments, one or more amplification reactions can be carried out in the presence of a blocking agent to increase specific amplification of the target nucleic acid. Such an agent can suppress background noise generated during amplification, increase specific amplification of one or more target nucleic acids, and/or improve the quality of amplification (e.g., possibly by improving the efficiency of amplification).

Blocking agents can be employed in any amplification reaction, for example, where a genomic DNA sample is being preamplified or amplified. Genomic DNA contains repetitive nucleotide sequences to which primers may non-specifically hybridize, which may increase background noise and compete with target nucleic acids for primers. The inclusion of a blocking agent in the amplification reaction mixture increases specific amplification of the target nucleic acid. In various embodiments, the increase in specific amplification can be about 10 percent, about 25 percent, about 50 percent, about 75 percent, about 100 percent, about 150 percent, about 200 percent, about 250 percent, about 300 percent, about 350 percent, about 400 percent, about 450 percent, or about 500 percent of the amplification observed in the absence of blocking agent. Without being bound by a particular theory, it is believed that the blocking may act by hybridizing to repetitive sequences in the genomic DNA sample.

Blocking agents also find particular utility in multiplex amplification reactions using genomic DNA or other types of nucleic acid samples. In multiplex amplification, the presence of multiple primers in the amplification reaction mixture can increase signal attributable to non-specific hybridization of the multiple primers. The inclusion of a blocking agent may suppress this signal.

In an illustrative embodiment, a nucleic acid blocking agent, such as tRNA, is employed as a blocking agent in an amplification reaction, such as, e.g., PCR. Other blocking agents can include degenerate oligonucleotide primers, repetitive DNA, BSA, or glycogen.

The blocking agent should present in an amount to increase specific amplification of the target nucleic acid. In certain embodiments, the blocking agent is present at a concentration in the range of about 0.1 μg/μL to about 40 μg/μL. In specific embodiments, the blocking agent concentration can be about 0.1, about 0.2, about 0.3, about 0.4, about 0.5, about 0.6, about 0.7, about 1, about 2, about 3, about 4, about 5, about 6, about 7, about 8, about 9, about 10, about 15, about 20, about 25, about 30, about 35, or about 40 μg/μL of the preamplification or amplification reaction mixture or can be any range having any of these values as endpoints (e.g., about 1 μg/μL to about 5 μg/μL). Suitable amounts can be also determined empirically, as shown in Example 5.

In an illustrative embodiment, tRNA is employed as a blocking agent at a concentration in the range of about 1 μg/μL to about 5 μg/μL, e.g., about 2 or 3 μg/μL.

### Labeling Strategies

Any suitable labeling strategy can be employed in the methods of the invention. Where the assay mixture is aliquoted, and each aliquot is analyzed for presence of a single amplification product, a universal detection probe can be employed in the amplification mixture. In particular embodiments, real-time PCR detection can be carried out using a universal qPCR probe. Suitable universal qPCR probes include double-stranded DNA dyes, such as SYBR Green, Pico Green (Molecular Probes, Inc., Eugene, Oreg.), ethidium bromide, and the like (see Zhu et al., 1994, Anal. Chem. 66:1941-48). Suitable universal qPCR probes also include sequence-specific probes that bind to a nucleotide sequence present in all amplification products. Binding sites for such probes can be conveniently introduced into the tagged target nucleic acids during preamplification (in embodiments employing preamplification) and/or into amplification products during amplification.

Alternatively, one or more target-specific qPCR probes (i.e., specific for a target nucleotide sequence to be detected) is employed in the amplification mixtures to detect amplification products. Target-specific probes could be useful, e.g., when only a few target nucleic acids are to be detected in a large number of samples. For example, if only three targets were to be detected, a target-specific probe with a different fluorescent label for each target could be employed. By judicious choice of labels, analyses can be conducted in which the different labels are excited and/or detected at different wavelengths in a single reaction. See, e.g., Fluorescence Spectroscopy (Pesce et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook of Fluorescent Probes and Research Chemicals, Molecular Probes, Eugene (1992).

### Matrix-Type Microfluidic Devices:

In certain embodiments, any of the methods of the invention can be carried out using a matrix-type microfluidic device. A matrix-type microfluidic device is one that allows the simultaneous combination of a plurality of substrate solutions with reagent solutions in separate isolated reaction chambers. It will be recognized, that a substrate solution can comprise one or a plurality of substrates and a reagent solution can comprise one or a plurality of reagents. For example, the microfluidic device can allow the simultaneous pair-wise combination of a plurality of different amplification primers and samples. In certain embodiments, the device is configured to contain a different combination of primers and samples in each of the different chambers. In various embodiments, the number of separate reaction chambers can be greater than 50, usually greater than 100, more often greater than 500, even more often greater than 1000, and sometimes greater than 5000, or greater than 10,000.

In certain embodiments, the matrix-type microfluidic device can be a Digital Array microfluidic device, that is adapted to perform digital amplification. Such devices can have integrated channels and valves that partition mixtures of sample and reagents into nanolitre volume reaction chambers. In some embodiments, the Digital Array microfluidic device is fabricated, at least in part, from an elastomer. Illustrative Digital Array microfluidic devices are described in copending U.S. patent applications owned by Fluidigm, Inc. (see, e.g., U.S. Patent Publication No. 20090239308, published Sep. 24, 2009) (see, e.g., FIG. 5).

As illustrated in FIG. 5, the nanofluidic biochip, also referred to as a digital array, includes a carrier 110, that can be made from materials providing suitable mechanical support for the various elements of the nanofluidic biochip. As an example, the biochip can be made using an elastomeric polymer. In one illustrative embodiments, the outer portion of the biochip has the same footprint as a standard 384-well microplate and enables stand-alone valve operation. As described below, in one embodiment, there are 12 input ports corresponding to 12 separate sample inputs to the chip. The biochip has 12 panels 105 and each of the 12 panels contains 765 6 nl reaction chambers with a total volume of 4.59 μL per panel. Microfluidic channels 112 connect the various reaction chambers on the panels to fluid sources as described more fully below.

Pressure can be applied to accumulator 120 in order to open and close valves connecting the reaction chambers to fluid sources. As illustrated in FIG. 5, 12 inlets 122 are provided for loading of the sample reagent mixture. 48 inlets 122 are used in some applications to provide a source for reagents that are supplied to the biochip when pressure is applied to accumulator 120. In applications in which reagents are not utilized, inlets 122 and reagent side accumulator 120 may not be used. Additionally, two inlets 132 are provided in the embodiment illustrated in FIG. 5 to provide hydration to the biochip. Hydration inlets 132 are in fluid communication with the biochip to facilitate the control of humidity associated with the reaction chambers. As will be understood to one of skill in the art, some elastomeric materials utilized in the fabrication of the biochip are gas permeable, allowing evaporated gases or vapor from the reaction chambers to pass through the elastomeric material into the surrounding atmosphere. In a particular embodiment, fluid lines located at peripheral portions of the biochip provide a shield of hydration liquid, for example, a buffer or master mix, at peripheral portions of the biochip surrounding the panels of reaction chambers, thus reducing or preventing evaporation of liquids present in the reaction chambers. Thus, humidity at peripheral portions of the biochip can be increased by adding a volatile liquid, for example water, to hydration inlets 132. In a specific embodiment, a first inlet is in fluid communication with the hydration fluid lines surrounding the panels on a first side of the biochip and the second inlet is in fluid communication with the hydration fluid lines surrounding the panels on the other side of the biochip.

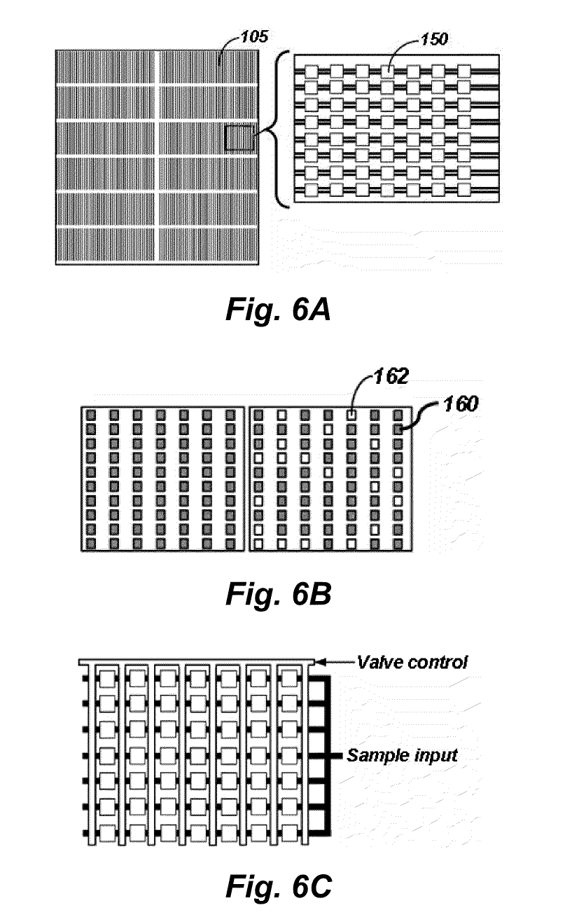

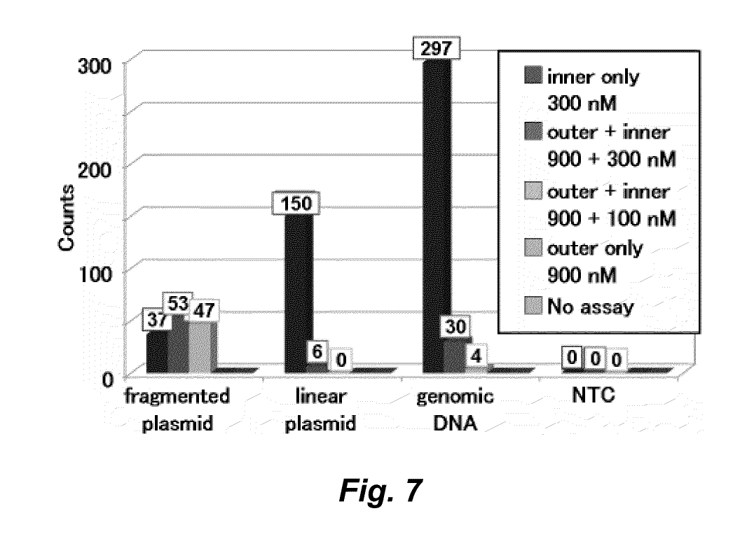

FIGS. 6A-6D are simplified diagrams of portion of the nanofluidic biochip illustrated in FIG. 5. FIG. 6A illustrates the 12 panels 105, each of the panels including a number of reaction chambers. As shown therein a number of reaction chambers 150 are contained in a panel. The reaction chambers 150 are spaced on 200 μm centers as illustrated. FIG. 6B illustrates a fluorescence image of a portion of a panel. The left side of the illustration is a control section, with all the reaction chambers illustrated as dark. The right side of the illustration shows how in a typical experiment, many of the reaction chambers are dark 160, generating no significant fluorescent emission. However, a portion of the reaction chambers have fluorescent emission, indicating a “positive” reaction chamber 162. As illustrated in FIG. 6C, sample channels run left to right connecting individual reaction chambers and control channels run top to bottom in the lower layer. Upon pressurization of the control channels, a thin membrane between layers closes off the sample channels to isolate individual reaction chambers. The valves partition individual chambers that are kept closed during the PCR experiment.

While the Digital Array microfluidic devices are well-suited for carrying out the digital amplification methods described herein, one of ordinary skill in the art would recognize many variations and alternatives to these devices. The microfluidic device which is the 12.765 Dynamic Array commercially available from Fluidigm Corp. (South San Francisco, Calif.), includes 12 panels, each having 765 reaction chambers with a volume of 6 mL per reaction chamber. However, this geometry is not required for the digital amplification methods described herein. The geometry of a given Digital Array microfluidic device will depend on the particular application. Additional description related to devices suitable for use in the methods described herein is provided in U.S. Patent Application Publication No. 2005/0252773, incorporated herein by reference for its disclosure of Digital Array microfluidic devices.

Fabrication methods using elastomeric materials and methods for design of devices and their components have been described in detail in the scientific and patent literature. See, e.g., Unger et al. (2000) Science 288:113-116; U.S. Pat. Nos. 6,960,437 (Nucleic acid amplification utilizing microfluidic devices); 6,899,137 (Microfabricated elastomeric valve and pump systems); 6,767,706 (Integrated active flux microfluidic devices and methods); 6,752,922 (Microfluidic chromatography); 6,408,878 (Microfabricated elastomeric valve and pump systems); 6,645,432 (Microfluidic systems including three-dimensionally arrayed channel networks); U.S. Patent Application Publication Nos. 2004/0115838; 2005/0072946; 2005/0000900; 2002/0127736; 2002/0109114; 2004/0115838; 2003/0138829; 2002/0164816; 2002/0127736; and 2002/0109114; PCT Publication Nos. WO 2005/084191; WO 05/030822A2; and WO 01/01025; Quake & Scherer (2000) Science 290: 1536-40; Unger et al. (2000) Science 288: 113-116; Thorsen et al. (2002) Science 298: 580-584; Chou et al. (2000) Biomedical Microdevices 3: 323-330; Liu et al. (2003) Analyt. Chem. 75: 4718-4723, Hong et al. (2004) Nat. Biotechnol., 22: 435-439.

According to certain embodiments of the invention, the detection and/or quantification of one or more target nucleic acids from one or more samples may generally be carried out on a matrix-type microfluidic device by obtaining a sample, optionally pre-amplifying the sample, and distributing the optionally pre-amplified sample, or aliquots thereof, into reaction chambers of a microfluidic device containing the appropriate buffers, primers, optional probe(s), and enzyme(s), subjecting these mixtures to amplification, and querying the aliquots for the presence of amplified target nucleic acids. The sample aliquots may have a volume of in the range of about 1 picoliter to about 500 nanoliters, more often in the range of about 100 picoliters to about 20 nanoliters, even more often in the range of about 1 nanoliter to about 20 nanoliters, and most often in the range of about 5 nanoliters to about 15 nanoliters.

In certain embodiments, multiplex detection is carried out in individual amplification mixture, e.g., in individual reaction chambers of a matrix-type microfluidic device, which can be used to further increase the number of samples and/or targets that can be analyzed in a single assay or to carry out comparative methods, such as comparative genomic hybridization (CGH).

In specific embodiments, the assay usually has a dynamic range of at least 3 orders of magnitude, more often at least 4, at least 5, at least 6, at least 7, or at least 8 orders of magnitude.

### Removal of Undesired Reaction Components

It will be appreciated that reactions involving complex mixtures of nucleic acids in which a number of reactive steps are employed can result in a variety of unincorporated reaction components, and that removal of such unincorporated reaction components, or reduction of their concentration, by any of a variety of clean-up procedures can improve the efficiency and specificity of subsequently occurring reactions. For example, it may be desirable, in some embodiments, to remove, or reduce the concentration of preamplification primers prior to carrying out the amplification steps described herein.

In certain embodiments, the concentration of undesired components can be reduced by simple dilution. For example, preamplified samples can be diluted about 2-, 5-, 10-, 50-, 100-, 500-, 1000-fold prior to amplification to improve the specificity of the subsequent amplification step.

In some embodiments, undesired components can be removed by a variety of enzymatic means.

In particular embodiments, clean-up includes selective immobilization of the desired nucleic acids. For example, desired nucleic acids can be preferentially immobilized on a solid support. In an exemplary embodiment, photo-biotin is attached to desired nucleic acid, and the resulting biotin-labeled nucleic acids immobilized on a solid support comprising an affinity-moiety binder such as streptavidin. Immobilized nucleic acids can be queried with probes, and non-hybridized and/or non-ligated probes removed by washing (See, e.g., Published P.C.T. Application WO 03/006677 and U.S. Ser. No. 09/931,285.)

### Data Output and Analysis

In certain embodiments, when the methods of the invention are carried out on a matrix-type microfluidic device, the data can be output as a heat matrix (also termed “heat map”). In the heat matrix, each square, representing a reaction chamber on the DA matrix, has been assigned a color value which can be shown in gray scale, but is more typically shown in color. In gray scale, black squares indicate that no amplification product was detected, whereas white squares indicate the highest level of amplification produce, with shades of gray indicating levels of amplification product in between. In a further aspect, a software program may be used to compile the data generated in the heat matrix into a more reader-friendly format.

### Applications

The methods of the invention are applicable to any technique aimed at detecting the presence or amount of one or more target nucleic acids in a nucleic acid sample. IN certain embodiments, the methods are particularly well suited to detect the presence and/or amount of one or more target nucleic acids found on short nucleic acids in a population of mixed long and short nucleic acids. Thus, for example, these methods are applicable to identifying the presence of particular polymorphisms (such as SNPs), alleles, or haplotypes, or chromosomal abnormalities, such as aneuploidies, amplifications, deletions, or translocations. The methods can be employed in genotyping, which can be carried out in a number of contexts, including diagnosis of genetic diseases or disorders, pharmacogenomics (personalized medicine), quality control in agriculture (e.g., for seeds or livestock), the study and management of populations of plants or animals (e.g., in aquaculture or fisheries management or in the determination of population diversity), or paternity or forensic identifications. The methods of the invention can be applied to the identification of sequences indicative of particular conditions or organisms in biological or environmental samples. For example, the methods can be used to identify pathogens, such as viruses, bacteria, and fungi). The methods can also be used to characterize environments or microenvironments, e.g., to characterize the microbial species in the human gut.

These methods can also be employed to determine DNA or RNA copy number. Determination of aberrant DNA copy number in genomic DNA is useful, for example, in the diagnosis and/or prognosis of genetic defects and diseases, such as cancer. Determination of RNA “copy number,” i.e., expression level is useful for expression monitoring of genes of interest, e.g., in different individuals, tissues, or cells under different conditions (e.g., different external stimuli or disease states) and/or at different developmental stages.

In addition, the methods can be employed to prepare nucleic acid samples for further analysis, such as, e.g., DNA sequencing.

Finally, nucleic acid samples can be tagged as a first step, prior to subsequent analysis, to reduce the risk that mislabeling or cross-contamination of samples will compromise the results. For example, any physician's office, laboratory, or hospital could tag samples immediately after collection, and the tags could be confirmed at the time of analysis. Similarly, samples containing nucleic acids collected at a crime scene could be tagged as soon as practicable, to ensure that the samples could not be mislabeled or tampered with. Detection of the tag upon each transfer of the sample from one party to another could be used to establish chain of custody of the sample.

### Kits

Kits according to the invention include one or more reagents useful for practicing one or more assay methods of the invention. A kit generally includes a package with one or more containers holding the reagent(s) (e.g., primers and/or probe(s)), as one or more separate compositions or, optionally, as admixture where the compatibility of the reagents will allow. In certain embodiments the kit includes a pair of inner primers and/ro a pair of outer primers to preferentially tag target sequences found on short nucleic acids in a population of mixed length nucleic acids. The kit can also, optionally, include other material(s) that may be desirable from a user standpoint, such as a buffer(s), a diluent(s), a standard(s), and/or any other material useful in sample processing, washing, or conducting any other step of the assay.

Kits according to the invention generally include instructions for carrying out one or more of the methods of the invention. Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

It is understood that the examples and embodiments described herein are for illustrative purposes only and that various modifications or changes in light thereof will be suggested to persons skilled in the art and are to be included within the spirit and purview of this application and scope of the appended claims.

In addition, all other publications, patents, and patent applications cited herein are hereby incorporated by reference in their entirety for all purposes.

### EXAMPLES

The following examples are offered to illustrate, but not to limit the claimed invention.

### Example 1

### Proof of Principle 1.2

### Targets:

The following targets were prepared: 1) Fragmented plasmid (3000 and 500 copies) win which plasmid DNA was restriction digested to yield independent fragments of either inner tagged primer pair (“target”) or one of outer primers; 2 Linear plasmid comprising a long fragment produced by linearlizing a plasmid with outer primers on same fragment as inner tagged primers, within 50 by (d=100); 3) gDNA comprising Long: outer primers on same fragment as inner tagged primers, within 100 by (d=150); and NTC.

### Protocol: Tagging of the Short:

Two cycles of tagging were performed with:

inner tagged primers only: Positive Control;

outer primer pair (900 nM) only: Negative control;

inner tagged primers (300 nM) and outer preimers (900 nM): Suppression;

inner tagged primers (100 nM) and outer primers (900 nM): Suppression; and

without assays: Negative control.

The mixture was treated with ExoSAP-it treatment to remove primers. Then 1 μL tag-specific primers were added to 10 μL PCR mix and TaqMan probe. A standard digital PCR was performed.

The expected results are illustrated in Table 1.

TABLE 1
Expected results.

| | | Outer + | Outer + | | |
|---|---|---|---|---|---|
| | Inner only | Inner | Inner 900 + | Outer only | |
| | 300 nM | 300 NM | 100 nM | 900 nM | No Assay |
| fragmented | + | + | + | − | − |
| plasmid | | | | | |
| fragmented | + | + | + | − | − |
| plasmid 1/6 | | | | | |
| linear | + | − | − | − | − |
| plasmid | | | | | |
| genomic | + | − | − | − | − |
| DNA | | | | | |
| NTC | − | − | − | − | − |

The actual measured results are shown in Table 2.

TABLE 2
Measured results.

| | | Outer + | | Outer | |
|---|---|---|---|---|---|
| | Inner only | Inner | Outer + Inner | only | No |
| | 300 nM | 300 NM | 900 + 100 nM | 900 nM | Assay |
| fragmented | 75 | 544 | 280 | 0 | 0 |
| plasmid | | | | | |
| fragmented | 37 | 53 | 47 | 0 | — |
| plasmid 1/6 | | | | | |
| linear plasmid | 150 | 6 | 0 | 0 | 0 |
| genomic DNA | 297 | 30 | 4 | 0 | 0 |
| NTC | 0 | 0 | 0 | 0 | 0 |

As expected the fragmented plasmid showed no effect of the presence of outer primers. Long DNA detection was suppressed almost completely by the presence of outer primers (linear plasmid, gDNA).

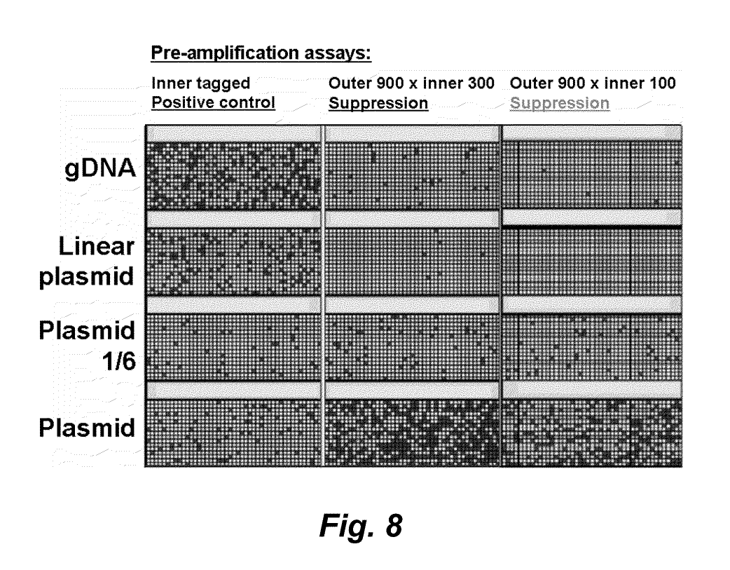

The data are summarized in FIG. 8. As can be seen, using this simple protocol, all controls were negative as expected. Suppression worked ≧90% without optimization. Less inner primer resulted in better suppression.

### Example 2

### Proof of Principle 1.3

A similar experiment was performed with the inner primer at 300 nM or 100 mM and the outer primer at 900 nM. This example provides an exemplary protocol for carrying out an assay method of the invention to genotype 16 SNPs in 144 samples using a 48.48 Dynamic Array available from Fluidigm Corporation, South San Francisco, Calif.

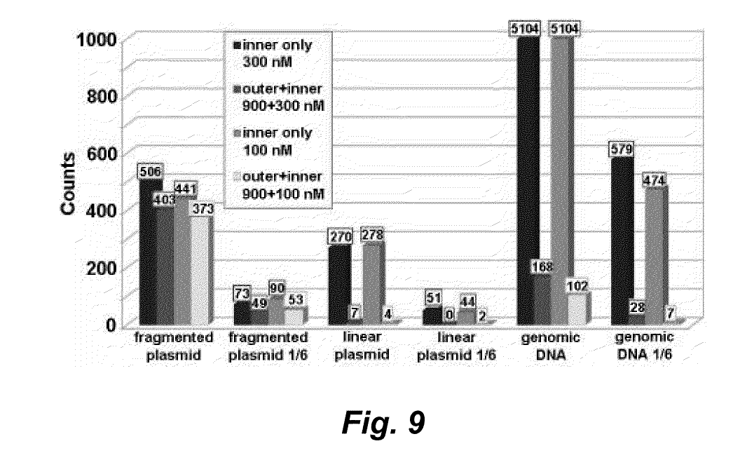

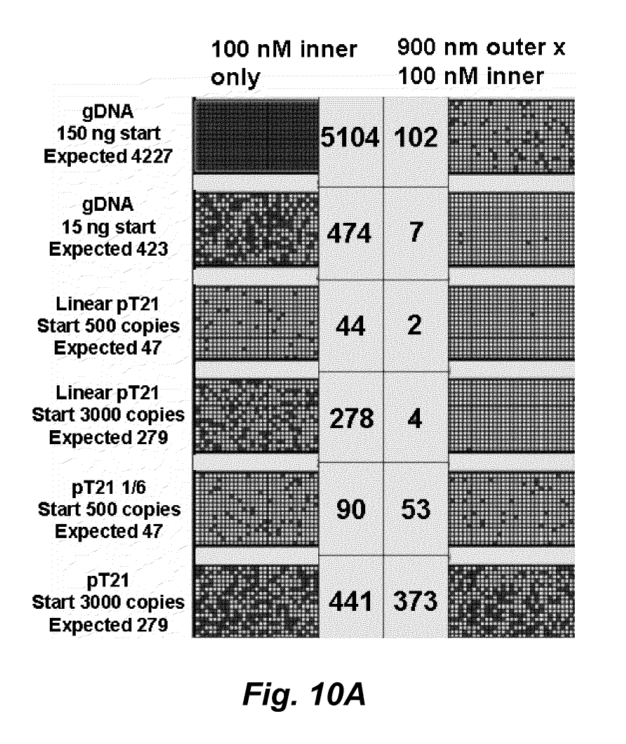

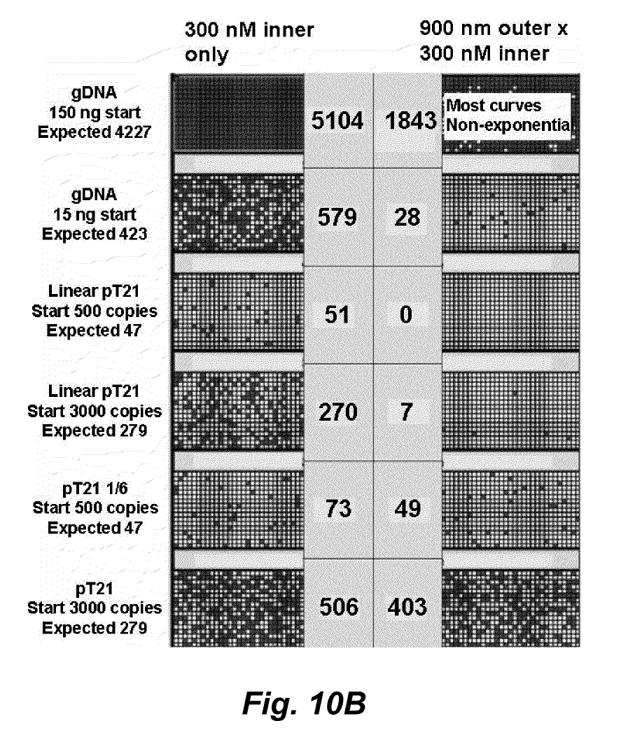

As shown in the graph (FIG. 9) and the heat map (FIG. 10) the fragmented plasmid showed no or minimal effect of the presence of the outer primers. Long DNA detection was suppressed almost completely by presence of outer primers (linear plasmid, gDNA). This worked at all concentrations. All controls were negative as expected.

### Example 3

### Proof of Principle 1.4

### Extra Cycles

The goal was to amplify tagged (short) product from two cycles in the same mix for another number of cycles. The annealing temperature (TA) was raised to prevent tagged primers from binding any sequence without tag.

The method was expected to increase the number of spots and thereby enable other applications such as FACts, hd-DID, and the like. The method was also expected to reduce background.

After two cycles of tagging (e.g., as above), the TA was increased to 72° C. from 60° C. At this temperature, only tagged primers will anneal, and only tagged product from first 2 cycles will amplify. Nine PCR cycles were performed at 95° C.-72°. The mixture was then treated with ExoSAP-IT, diluted, and analyzed in a digital chip.

The results are shown in Table 3.

TABLE 3
Results of extra amplification cycles.

| | | | | amplification |
|---|---|---|---|---|
| | | | | (compared to |
| | Inner | Outer and Inner | suppression | POC1.4) |
| fragmented | 2176 | 2211 | −2% | 430 |
| plasmid | | | | |
| linear | 1452 | 80 | 94% | 538 |
| plasmid | | | | |
| genomic DNA | 2371 | 121 | 95% | 409 |
| genomic DNA | 2416 | 119 | 95% | 417 |
| (inner 100 nM) | | | | |

### Example 4

### Proof of Principle 1.5

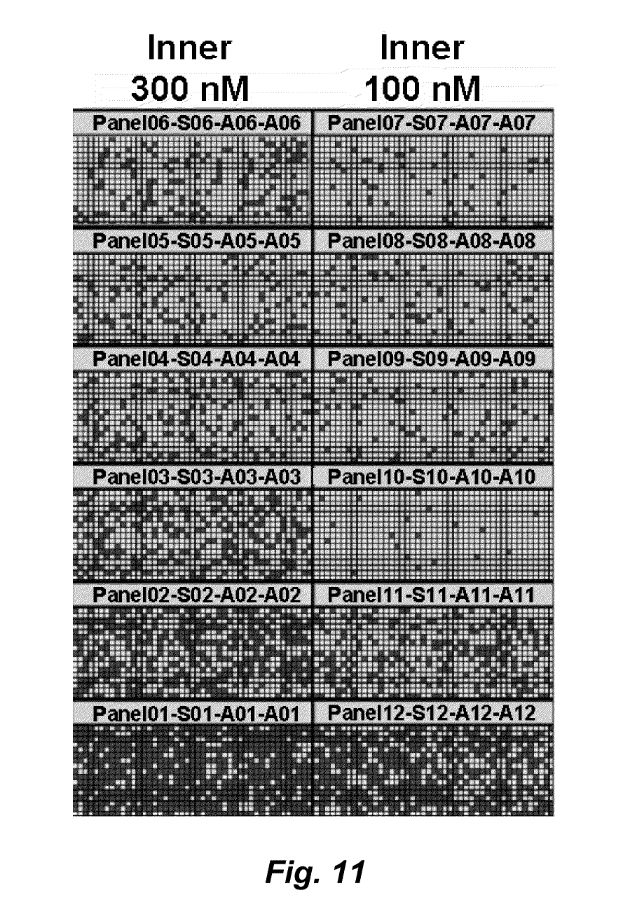

In another experiment, plasmid T21 was spiked into a gDNA background. The samples were plasmid T21 diluted in gDNA background at 1000, 333, 111, 37, 12, and 0 copies. Amplification was performed with inner primers at 300 nM and 100 nM and outer primers at 900 nM. The results are shown in the heat map in FIG. 11.

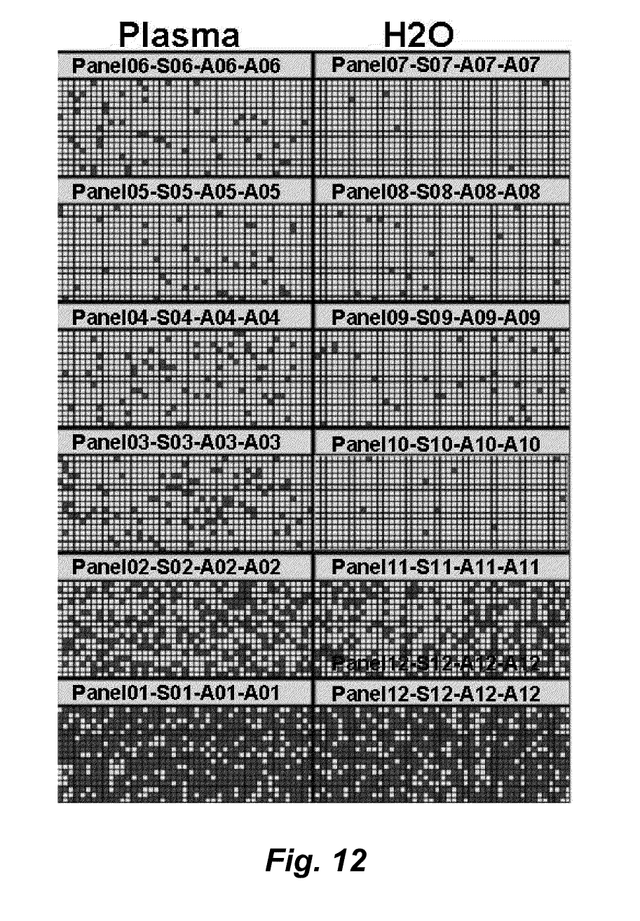

Plasmid T21 was then spiked into plasma and water (H₂O) backgrounds at 1000, 333, 111, 37, 12, and 0 copies. Amplification was performed with inner primers at 100 nM and outer primers at 900 nM. The results are shown in the heat map in FIG. 12, and summarized in Table 4.

TABLE 4
Summary of estimated and measured results of spiking experiments.
Total: 500 μL product; 0.465 μL of which per panel; total copies:
est count × 500/0.465 = 1075 × est count. Amplification: 11 cycles,
3rd cycle makes tagged ds --> 1024 X.

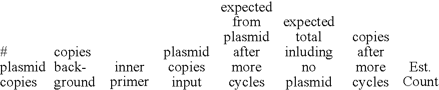

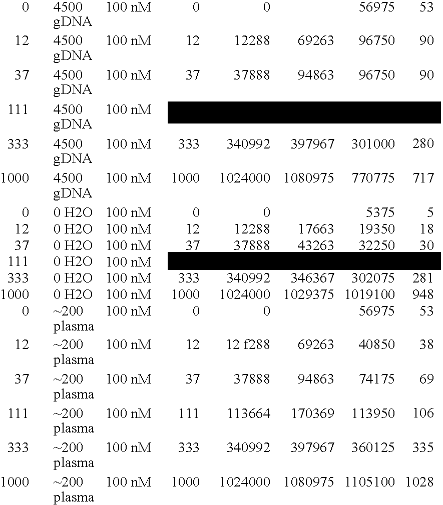

### Example 5

### Use of tRNA in Amplification of Genomic DNA

Human genomic DNA was preamplified using standard protocols on the GeneAmp PCR system 9700 (Applied Biosystems, CA) in a 25 μL reaction containing 1× PreAmp master mix (Applied Biosystems, CA), 900 nM primers, about 10 ng of DNA sample, and differing amounts of tRNA (transfer ribonucleic acid, from baker's yeast S. cerevisiae, Sigma Chemicals, cat no RS636-1mL). Samples were diluted and analyzed by digital PCR on a 12.765 Digital Array commercially available from Fluidigm Corp. (South San Francisco, Calif.). The thermal cycling protocol followed was similar to that reported in Qin J., Jones R C, Ramakrishnan R. (2008) Studying copy number variations using a nanofluidic platform Nucleic Acids Research, Vol. 36, No. 18 e116.

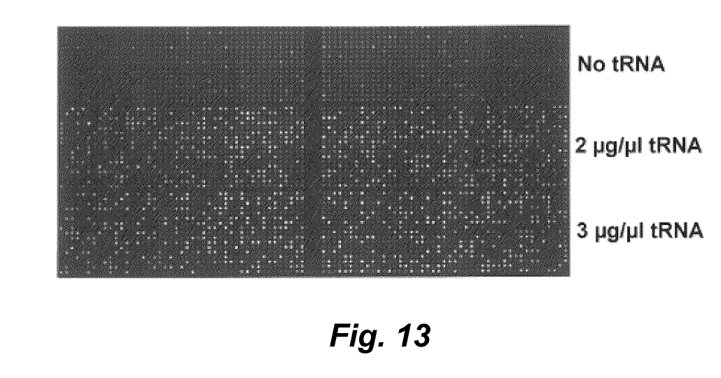

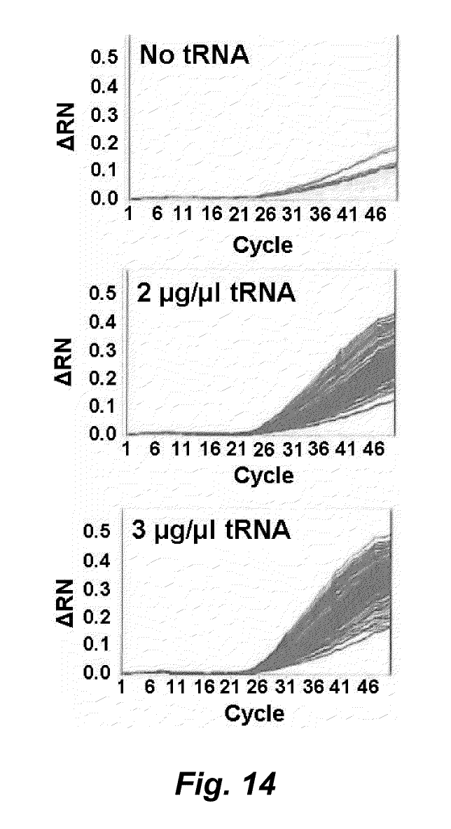

FIGS. 13 and 14 demonstrate that the addition of tRNA increases the intensity of the specific amplification signal, suppresses background, and improves the quality of specific amplification curves. Table 5, below, shows the increase in specific counts with the addition of tRNA.

TABLE 5
Increase in specific counts with addition of tRNA.

| Amount of tRNA | Counts* |
|---|---|
| None | 9 |
| 2 μg/μL | 290 |
| 3 μg/μL | 275 |

*Average number of signals per panel of 12.765 Digital Array

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology or in the relevant fields are intended to be within the scope of the appended claims.

1. A method for depleting a nucleic acid sample of non-target nucleic acids, the method comprising:
denaturing the sample nucleic acids in a reaction mixture;
contacting the denatured sample nucleic acids with at least one target-specific primer pair under suitable annealing conditions;
conducting a first cycle of extension of any annealed target-specific primer pairs by nucleotide polymerization; and
after the first cycle of extension, conducting a first cycle of nuclease digestion of single-stranded nucleic acid sequences in the reaction mixture.

2. The method of claim 1, additionally comprising:
after the first cycle of nuclease digestion, denaturing the nucleic acids in the reaction mixture;
contacting the denatured nucleic acids with at least one target-specific primer pair under suitable annealing conditions;
conducting a second cycle of extension of any annealed target-specific primer pairs by nucleotide polymerization; and
conducting a second cycle of nuclease digestion of single-stranded nucleic acid sequences in the reaction mixture.

3. The method of claim 1, wherein the same target-specific primer pair is used to prime each of the first and second cycles of extension.

4. The method of claim 2, wherein the nuclease is selected from the group consisting of a single strand-specific 3′ exonuclease, a single strand-specific endonuclease, and a single strand-specific 5′ exonuclease.

5. The method of claim 4, wherein the nuclease comprises E. coli Exonuclease I.

6. The method of claim 2, wherein the target-specific primers comprise dU, rather than dT, and dUTP, rather than dTTP, is present in the reaction mixture.

7. The method of claim 6, additionally comprising:
after second cycle of nuclease digestion, contacting the reaction mixture with E. coli Uracil-N-Glycosylase.

8. The method of claim 1, wherein said method is carried out using two or more target-specific primer pairs, wherein each primer pair is specific for a different target nucleotide sequence.

9. The method of claim 2, additionally comprising:
after the second cycle of nuclease digestion, denaturing the nucleic acids in the reaction mixture;
contacting the denatured nucleic acids with at least one tag specific primer pair under suitable annealing conditions; and
amplifying the corresponding tagged target nucleotide sequence.

10. A method for selective tagging of short nucleic acids comprising a short target nucleotide sequence (molecule) over longer nucleic acids comprising the same target nucleotide sequence, the method comprising:
denaturing sample nucleic acids in a reaction mixture, wherein the sample nucleic acids comprise long nucleic acids and short nucleic acids, each comprising the same target nucleotide sequence;
contacting the denatured sample nucleic acids with at least two target-specific primers or primer pairs under suitable annealing conditions, wherein the primer pairs comprise:
an inner primer or primer pair that can amplify the target nucleotide sequence on long and short nucleic acids, wherein each inner primer comprises a 5′ nucleotide tag; and
an outer primer or primer pair that amplifies the target nucleotide sequence on long nucleic acids, but not on short nucleic acids;
conducting a first cycle of extension of any annealed primer pairs by nucleotide polymerization;
after the first cycle of extension, denaturing the nucleic acids in the reaction mixture;
subjecting the reaction mixture to suitable annealing conditions; and conducting a second cycle of extension to produce at least one tagged target nucleotide sequence that comprises two nucleotide tags, one from each inner primer, with the target nucleotide sequence located between the nucleotide tags.

11. The method of claim 10, additionally comprising:
after the first cycle of extension, digesting single-stranded nucleic acid sequences in the reaction mixture.

12. The method of claim 10, additionally comprising:
after the second cycle of extension, digesting single-stranded nucleic acid sequences in the reaction mixture.

13. The method of claim 11, wherein the digestion is carried out by adding, to the reaction mixture, a nuclease selected from the group consisting of a single strand-specific 3′ exonuclease, single strand-specific endonuclease, and a single strand-specific 5′ exonuclease.

14. (canceled)

15. The method of claim 11, wherein said at least two target-specific primer pairs are protected against digestion with said nuclease.

16. The method of claim 11, additionally comprising:
after the digestion, adding additional quantities of said at least two target-specific primer pairs to the reaction mixture.

17. The method of claim 10, wherein:
after the first cycle of extension, any subsequent denaturation is carried out at a sufficiently low temperature to avoid denaturation of any extension product of the outer primer pair.

18. The method of claim 17, wherein the denaturation temperature is about 80° C. to about 85° C.

19. The method of claim 10, wherein the short nucleic acid fragments are less than about 300 nucleotides in length.

20. The method of claim 19, wherein the distance from each outer primer to the target nucleotide sequence is about 130 nucleotides or greater.

21. (canceled)

22. The method of claim 19, wherein the short nucleic acid fragments comprise fetal DNA, and the long nucleic acid fragments comprise maternal DNA.

23. The method of claim 22, wherein the sample comprises maternal plasma.

24. The method of claim 10, wherein the short nucleic acid fragments comprise tumor DNA, and the long nucleic acids comprise normal DNA.

25. The method of claim 24, wherein the sample comprises plasma from a cancer patient.

26. The method of claim 10, additionally comprising:
subjecting the reaction mixture to one or more cycles of amplification, wherein annealing is carried out at a sufficiently high temperature that the inner primers will only anneal to tagged target nucleotide sequences.

27. The method of claim 10, additionally comprising:
contacting the at least one tagged target nucleotide sequence with a tag-specific primer pair under suitable annealing conditions; and
amplifying or otherwise detecting and/or quantifying the tagged target nucleotide sequence.

28. The method of claim 27, additionally comprising quantifying the amount of said at least one tagged target nucleotide sequence produced by amplification.

29. The method of claim 28, wherein said quantifying comprises subjecting the tagged target nucleotide sequence(s) to digital amplification.

30. The method of claim 29, wherein the amplification of claim 27 comprises a preamplification that produces at least one target amplicon.

31. The method of claim 30, wherein said preamplification comprises amplifying a tagged reference nucleic acid to produce a reference amplicon.

32. The method of claim 31, wherein said digital amplification comprises:
distributing the preamplified target and reference amplicons into discrete digital amplification mixtures, wherein each digital amplification mixture, on average, includes no more than one amplicon per mixture; and
subjecting the digital amplification mixtures to amplification.

33. The method of claim 32, wherein said digital amplification comprises real-time PCR and/or endpoint PCR.

34. (canceled)

35. The method of claim 32, wherein said digital amplification comprises:
determining the number of reaction mixtures containing amplification product derived from a particular target amplicon;
determining the number of reaction mixtures containing amplification product derived from the reference amplicon; and
determining the copy number for each target amplicon relative to the reference amplicon.

36. The method of claim 35, wherein the target amplicon is derived from fetal DNA.

37. The method of claim 35, wherein the target amplicon is derived from tumor DNA.

38. The method of claim 10, wherein said method is carried out using at least one additional set of inner and outer target-specific primer pairs, when the set is specific for at least one additional target nucleotide sequence.

39. The method of claim 38, wherein the additional inner primer pair comprises 5′ nucleotide tags that are different from the 5′ nucleotide tags of claim 10.

40. The method of claim 38, wherein the additional inner primer pair comprises 5′ nucleotide tags that are the same as the 5′ nucleotide tags of claim 10.

41. The method of claim 40, wherein at least two different target nucleotide sequences that are tagged with the same tags are located on the same chromosome.

42. The method of claim 10, wherein said amplification is carried out in one or more compartment(s) of a microfluidic device.

43. The method of claim 42, wherein the microfluidic device is fabricated, at least in part, from an elastomeric material.

44. The method of claim 10 further comprising detecting and/or quantifying the tagged short target nucleic acid.

45. The method of claim 44, wherein the presence of a target amplicon is determined by ligase detection reaction (LDR), or by quantitative real-time polymerase chain reaction (qPCR).

46. (canceled)

47. The method of claim 44, wherein a universal qPCR probe is employed to detect target amplicon(s).

48. The method of claim 47, wherein the universal qPCR probe comprises a double-stranded DNA-binding dye.

49. The method of claim 10, wherein one or more target-specific qPCR probes is employed to detect target amplicon(s).

50. The method of claim 10, wherein the presence of a target amplicon is detected using a fluorogenic nuclease assay.

51. The method of claim 10, wherein the presence of a target amplicon is detected using a dual-labeled fluorogenic hydrolysis oligonucleotide probe.

52. The method of claim 10, wherein the method is performed to determine genotypes at loci corresponding to the target nucleotide sequence.

53. The method of claim 10, wherein the method is performed to determine copy number at loci corresponding to the target nucleotide sequence.

54. The method of claim 53, wherein the method is performed to determine the presence or absence of fetal aneuploidy.

55. The method of claim 10, wherein the method is performed to prepare target nucleotide sequence(s) for sequencing.

56. The method of claim 1, wherein the sample comprises a genomic DNA sample.

57. The method of claim 56, wherein one or more amplification cycles are conducted in the presence of an amount of a blocking agent that is sufficient to increase specific amplification of the target nucleic acid.

58. The method of claim 57, wherein the blocking agent comprises a nucleic acid blocking agent that hybridizes to repetitive sequences in the genomic DNA sample.

59. The method of claim 57, wherein the blocking agent is selected from the group consisting of tRNA, degenerate oligonucleotide primers, repetitive DNA, bovine serum albumin (BSA), and glycogen.

60-61. (canceled)

62. A method of increasing the specific amplification of a target nucleic acid from a genomic DNA sample, the method comprising conducting the amplification in the presence of an amount of a blocking agent sufficient to increase specific amplification of the target nucleic acid.

63. The method of claim 62, wherein the blocking agent comprises a nucleic acid blocking agent that hybridizes to repetitive sequences in the genomic DNA sample.

64. A method of increasing the specific amplification of a plurality of target nucleic acids in a multiplex amplification reaction, the method comprising conducting the amplification in the presence of an amount of a blocking agent sufficient to increase specific amplification of the target nucleic acid.

65. (canceled)

66. The method of claim 62, wherein the blocking agent is selected from the group consisting of tRNA, degenerate oligonucleotide primers, repetitive DNA, bovine serum albumin (BSA), and glycogen.

67-68. (canceled)


SEQUENCE LISTING (1 sequence)

SEQ ID NO: 1 - protein, 8 aa, Artificial
  Artificial peptide epitope for antibody
DYKDDDDK